SEQUENCE LISTING (24 sequences)

SEQ ID NO: 1 - DNA, 1353 bp, Artificial Sequence
  Description of Artificial Sequence Synthetic polynucleotide
gaggtgcagctggtggaatctggggggggactggtgcagcctggcgggtcactgagactgtcctgtgccgcttctggctactcttttaccggccactggatgaactgggtgcgacaggcaccaggcaagggactggagtgggtcggaatgatccatccttctgacagtgaaacacggtacaatcagaagttcaaagaccggttcaccatttcagtggataagagcaaaaacactctgtacctccagatgaacagcctgagggccgaggacaccgctgtctactattgcgccagaggcatctacttctatggaaccacatactttgattattggggacagggcactctggtgaccgtcagctcctagagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggttga

SEQ ID NO: 2 - DNA, 642 bp, Artificial Sequence
  Description of Artificial Sequence Synthetic polynucleotide
gacattcagatgactcagagcccttcatctctgagtgcctcagtgggggaccgggtcaccatcacatgcagagccagcaaaacaatttccaagtacctggcttggtatcagcagaagcccggcaaagcacctaagctgctgatctacagcggcagcaccctccagtctggagtgccctccaggttctctggcagtgggtcaggaacagactttactctgaccatcagcagcctccagccagaggatttcgctacttactattgtcagcagcacaacgaatatcccctgacatttggccaggggactaaagtcgagatcaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 3 - DNA, 363 bp, Artificial Sequence
  Description of Artificial Sequence Synthetic polynucleotide
gaggtgcagctggtggaatctggggggggactggtgcagcctggcgggtcactgagactgtcctgtgccgcttctggctactcttttaccggccactggatgaactgggtgcgacaggcaccaggcaagggactggagtgggtcggaatgatccatccttctgacagtgaaacacggtacaatcagaagttcaaagaccggttcaccatttcagtggataagagcaaaaacactctgtacctccagatgaacagcctgagggccgaggacaccgctgtctactattgcgccagaggcatctacttctatggaaccacatactttgattattggggacagggcactctggtgaccgtcagctcc

SEQ ID NO: 4 - DNA, 291 bp, Artificial Sequence
  Description of Artificial Sequence Synthetic polynucleotide
tagagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaa

SEQ ID NO: 5 - DNA, 48 bp, Artificial Sequence
  Description of Artificial Sequence Synthetic oligonucleotide
gttgagcccaaatcttgtgacaaaactcacacatgcccaccgtgccca

SEQ ID NO: 6 - DNA, 330 bp, Artificial Sequence
  Description of Artificial Sequence Synthetic polynucleotide
gcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaa

SEQ ID NO: 7 - DNA, 321 bp, Artificial Sequence
  Description of Artificial Sequence Synthetic polynucleotide
gggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggttga

SEQ ID NO: 8 - DNA, 321 bp, Artificial Sequence
  Description of Artificial Sequence Synthetic polynucleotide
gacattcagatgactcagagcccttcatctctgagtgcctcagtgggggaccgggtcaccatcacatgcagagccagcaaaacaatttccaagtacctggcttggtatcagcagaagcccggcaaagcacctaagctgctgatctacagcggcagcaccctccagtctggagtgccctccaggttctctggcagtgggtcaggaacagactttactctgaccatcagcagcctccagccagaggatttcgctacttactattgtcagcagcacaacgaatatcccctgacatttggccaggggactaaagtcgagatcaag

SEQ ID NO: 9 - DNA, 321 bp, Artificial Sequence
  Description of Artificial Sequence Synthetic polynucleotide
cgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 10 - protein, 450 aa, Artificial Sequence
  Description of Artificial Sequence Synthetic polypeptide
EVQLVESGGGLVQPGGSLRLSCAASGYSFTGHWMNWVRQAPGKGLEWVGMIHPSDSETRYNQKFKDRFTISVDKSKNTLYLQMNSLRAEDTAVYYCARGIYFYGTTYFDYWGQGTLVTVSSXSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 11 - protein, 214 aa, Artificial Sequence
  Description of Artificial Sequence Synthetic polypeptide
DIQMTQSPSSLSASVGDRVTITCRASKTISKYLAWYQQKPGKAPKLLIYSGSTLQSGVPSRFSGSGSGTDFTLTISSLQPEDFATYYCQQHNEYPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 12 - protein, 121 aa, Artificial Sequence [sequence omitted]

SEQ ID NO: 13 - protein, 97 aa, Artificial Sequence
  Description of Artificial Sequence Synthetic polypeptide
XSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKK

SEQ ID NO: 14 - protein, 16 aa, Artificial Sequence
  Description of Artificial Sequence Synthetic peptide
VEPKSCDKTHTCPPCP

SEQ ID NO: 15 - protein, 110 aa, Artificial Sequence
  Description of Artificial Sequence Synthetic polypeptide
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 16 - protein, 106 aa, Artificial Sequence [sequence omitted]

SEQ ID NO: 17 - protein, 107 aa, Artificial Sequence [sequence omitted]

SEQ ID NO: 18 - protein, 107 aa, Artificial Sequence [sequence omitted]

SEQ ID NO: 19 - protein, 10 aa, Artificial Sequence
  Description of Artificial Sequence Synthetic peptide
GYSFTGHWMN

SEQ ID NO: 20 - protein, 17 aa, Artificial Sequence
  Description of Artificial Sequence Synthetic peptide
MIHPSDSETRYNQKFKD

SEQ ID NO: 21 - protein, 12 aa, Artificial Sequence
  Description of Artificial Sequence Synthetic peptide
GIYFYGTTYFDY

SEQ ID NO: 22 - protein, 11 aa, Artificial Sequence
  Description of Artificial Sequence Synthetic peptide
RASKTISKYLA

SEQ ID NO: 23 - protein, 7 aa, Artificial Sequence
  Description of Artificial Sequence Synthetic peptide
SGSTLQS

SEQ ID NO: 24 - protein, 9 aa, Artificial Sequence
  Description of Artificial Sequence Synthetic peptide
QQHNEYPLT

(19) United States
(12) Patent Application Publication
(10) Pub. No.: US 2016/0324981 A1
(43) Pub. Date: Nov. 10, 2016

(54) LIVER X RECEPTOR AGONISTS AND USES THEREOF

(71) Applicant: The California Institute for Biomedical Research, La Jolla, CA (US)

(72) Inventors: Stephanie PINKERTON, San Diego, CA (US); Matthew S. TREMBLAY, San Diego, CA (US); Arnab K. CHATTERJEE, San Diego, CA (US); Bo CHENG, San Diego, CA (US); Reyna K. V. LIM, San Diego, CA (US); Shan YU, San Diego, CA (US); Peter G. SCHULTZ, La Jolla, CA (US)

(21) Appl. No.: 15/147,863

(22) Filed: May 5, 2016

Related U.S. Application Data
(60) Provisional application No. 62/158,726, filed on May 8, 2015.

(51) Int. Cl. A61K 47/48; C07K 16/28

(52) U.S. Cl. CPC A61K 47/48561; C07K 16/2845

(57) ABSTRACT

Disclosed herein are antibody drug conjugates having an antibody or antibody fragment that binds a cell surface molecule on a target cell, wherein the target cell is a lymphocyte and a therapeutic agent that has a therapeutic effect in a subject in need thereof. Further disclosed herein are antibody drug conjugates having an antibody or antibody fragment that binds a cell surface molecule on a target cell; and a therapeutic agent that binds an intracellular moiety of the target cell. These antibody drug conjugates may be used for treating cardiovascular diseases.

### CROSS-REFERENCE TO RELATED APPLICATION

The present application claims priority to U.S. Provisional Application No. 62/158,726 filed May 8, 2015, the entire content of which is incorporated by reference in its entirety.

### SEQUENCE LISTING

The instant application contains a Sequence Listing which has been submitted electronically in ASCII format and is hereby incorporated by reference in its entirety. Said ASCII copy, created on May 5, 2016, is named 41135-744-201_SL.txt and is 21,519 bytes in size.

### BACKGROUND OF THE INVENTION

Liver X receptor (LXR) agonists have been explored as potential treatments for atherosclerosis and other diseases based on their ability to induce reverse cholesterol transport and suppress inflammation. However, this therapeutic potential has been hindered by on-target adverse effects in the liver mediated by excessive lipogenesis.

With the recent FDA approval of two antibody-drug conjugates (ADCs), namely brentuximab vedotin for Hodgkin's lymphoma and ado-trastuzumab emtansine for Her2 positive breast cancer, along with the increasing number of such agents currently in clinical trials, ADCs have garnered significant interest in oncology. ADCs can deliver cytotoxic agents selectively to tissues by binding to antigens highly expressed on specific cell types, followed by internalization and subsequent intracellular drug release. The net result is that the small-molecule payloads are delivered directly to the cell type of interest, reducing unwanted side effects resulting from off-target and/or off-tissue toxicity. Despite significant progress in the development of ADCs, few indications outside oncology have been explored.

### SUMMARY OF THE INVENTION

In one aspect, provided herein is an antibody drug conjugate comprising (a) a liver X receptor agonist and (b) an antibody that binds a cell surface molecule that is not expressed on a hepatocyte. In some embodiments, the cell surface molecule is expressed on a target cell selected from a monocyte, macrophage, foam cell, T cell, platelet, endothelial cell, endothelial cell progenitor, and vascular smooth muscle cell. In some embodiments, the target cell comprises a macrophage. In some embodiments, the cell surface molecule is selected from CD11a, CD11b, CD11c, CD163, CD64, CD68, CD80, and CD86. In some embodiments, the cell surface molecule is CD11a. In some embodiments, the antibody has a heavy chain variable domain having at least about 80% sequence identity to SEQ ID NO. 12. In some embodiments, the antibody has a heavy chain variable domain that differs from SEQ ID NO. 12 by less than about 5 amino acid residues. In some embodiments, the antibody comprises a heavy chain CDR1 sequence identical to SEQ ID NO. 19. In some embodiments, the antibody comprises a heavy chain CDR1 protein sequence that differs from SEQ ID NO. 19 by 3, 2 or 1 amino acid residues. In some embodiments, the antibody comprises a heavy chain CDR2 protein sequence identical to SEQ ID NO. 20. In some embodiments, the antibody comprises a heavy chain CDR2 protein sequence that differs from SEQ ID NO. 20 by 3, 2 or 1 amino acid residues. In some embodiments, the antibody comprises a heavy chain CDR3 protein sequence identical to SEQ ID NO. 21. In some embodiments, the antibody comprises a heavy chain CDR3 protein sequence that differs from SEQ ID NO. 21 by 3, 2 or 1 amino acid residues. In some embodiments, the antibody comprises a light chain CDR1 protein sequence identical to SEQ ID NO. 22. In some embodiments, the antibody comprises a light chain CDR1 protein sequence that differs from SEQ ID NO. 22 by 3, 2 or 1 amino acid residues. In some embodiments, the antibody comprises a light chain CDR2 protein sequence identical to SEQ ID NO. 23. In some embodiments, the antibody comprises a light chain CDR2 protein sequence that differs from SEQ ID NO. 23 by 3, 2 or 1 amino acid residues. In some embodiments, the antibody comprises a light chain CDR3 protein sequence identical to SEQ ID NO. 24. In some embodiments, the antibody comprises a light chain CDR3 protein sequence that differs from SEQ ID NO. 24 by 3, 2 or 1 amino acid residues.

In some embodiments, the liver X receptor agonist binds to liver X receptor alpha within a cell expressing the cell surface molecule. In some embodiments, the liver X receptor agonist binds to liver X receptor beta within a cell expressing the cell surface molecule. In some embodiments, the antibody is chemically linked to (a) the liver X receptor agonist or (b) a moiety chemically attached to the liver X receptor agonist. In some embodiments, the antibody comprises an unnatural amino acid chemically linked to (a) the liver X receptor agonist or (b) the moiety chemically attached to the liver X receptor agonist. In some embodiments, the antibody has a heavy chain sequence having at least about 90% sequence identity to SEQ ID NO. 10. In some embodiments, the antibody has a heavy chain sequence that differs from SEQ ID NO. 10 by less than about 5 amino acid residues. In some embodiments, the unnatural amino acid is incorporated at position A122 of the antibody. In some embodiments, the unnatural amino acid is para-acetylphenylalanine (pAcF). In some embodiments, the antibody is chemically linked to the moiety chemically attached to the liver X receptor agonist. In some embodiments, the moiety comprises a cleavage site recognized by a human protease. In some embodiments, the moiety comprises a cleavage site comprising a peptide. In some embodiments, the antibody is chemically linked to (a) the liver X receptor agonist or (b) the moiety chemically attached to the liver X receptor agonist via N-hydroxysuccinamide ester chemistry. In some embodiments, the liver X receptor agonist comprises compound 3 or a derivative or conjugate thereof. In some embodiments, the liver X receptor agonist comprises compound 10 or a derivative or conjugate thereof. In some embodiments, the conjugate of compound 10 is R in FIG. 3A. In some embodiments, the liver X receptor agonist has the structure R as shown in FIG. 3A, and wherein the liver X receptor agonist is linked to the unnatural amino acid para-acetylphenylalanine in the antibody. In some embodiments, the antibody drug conjugate has the structure I:

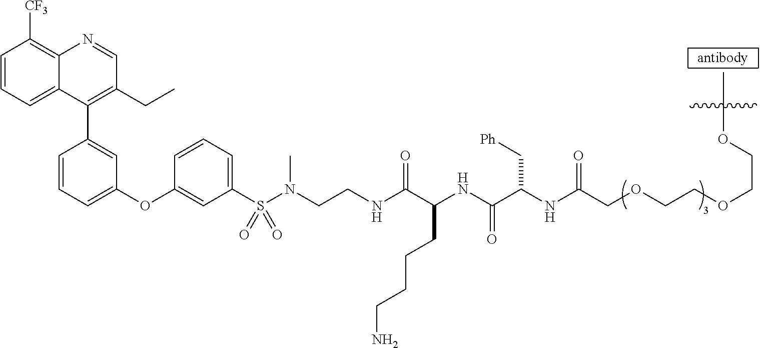

In another aspect, provided herein is an antibody drug conjugate comprising (a) a liver X receptor agonist and (b) an antibody that binds to an integrin expressed on a surface of a leukocyte. The antibody drug conjugate of claim 23, wherein the integrin is CD11a. In some embodiments, the antibody has a heavy chain variable domain having at least about 80% sequence identity to SEQ ID NO. 12. In some embodiments, the antibody has a heavy chain variable domain that differs from SEQ ID NO. 12 by less than about 5 amino acid residues. In some embodiments, the antibody comprises a heavy chain CDR1 sequence identical to SEQ ID NO. 19. In some embodiments, the antibody comprises a heavy chain CDR1 protein sequence that differs from SEQ ID NO. 19 by 3, 2 or 1 amino acid residues. In some embodiments, the antibody comprises a heavy chain CDR2 protein sequence identical to SEQ ID NO. 20. In some embodiments, the antibody comprises a heavy chain CDR2 protein sequence that differs from SEQ ID NO. 20 by 3, 2 or 1 amino acid residues. In some embodiments, the antibody comprises a heavy chain CDR3 protein sequence identical to SEQ ID NO. 21. In some embodiments, the antibody comprises a heavy chain CDR3 protein sequence that differs from SEQ ID NO. 21 by 3, 2 or 1 amino acid residues. In some embodiments, the antibody comprises a light chain CDR1 protein sequence identical to SEQ ID NO. 22. In some embodiments, the antibody comprises a light chain CDR1 protein sequence that differs from SEQ ID NO. 22 by 3, 2 or 1 amino acid residues. In some embodiments, the antibody comprises a light chain CDR2 protein sequence identical to SEQ ID NO. 23. In some embodiments, the antibody comprises a light chain CDR2 protein sequence that differs from SEQ ID NO. 23 by 3, 2 or 1 amino acid residues. In some embodiments, the antibody comprises a light chain CDR3 protein sequence identical to SEQ ID NO. 24. In some embodiments, the antibody comprises a light chain CDR3 protein sequence that differs from SEQ ID NO. 24 by 3, 2 or 1 amino acid residues.

In some embodiments, the antibody is chemically linked to (a) the liver X receptor agonist or (b) a moiety chemically attached to the liver X receptor agonist. In some embodiments, the antibody comprises an unnatural amino acid chemically linked to (a) the liver X receptor agonist or (b) the moiety chemically attached to the liver X receptor agonist. In some embodiments, the antibody has a heavy chain sequence having at least about 90% sequence identity to SEQ ID NO. 10. In some embodiments, the antibody has a heavy chain sequence that differs from SEQ ID NO. 10 by less than about 5 amino acid residues. In some embodiments, the unnatural amino acid is incorporated at position A122 of the antibody. In some embodiments, the unnatural amino acid is para-acetylphenylalanine (pAcF). In some embodiments, the antibody is chemically linked to the moiety chemically attached to the liver X receptor agonist. In some embodiments, the moiety comprises a cleavage site recognized by a human protease. In some embodiments, the moiety comprises a cleavage site comprising a peptide. In some embodiments, the antibody is chemically linked to (a) the liver X receptor agonist or (b) the moiety chemically attached to the liver X receptor agonist via N-hydroxysuccinamide ester chemistry. In some embodiments, the liver X receptor agonist comprises compound 3 or a derivative or conjugate thereof. In some embodiments, the liver X receptor agonist comprises compound 10 or a derivative or conjugate thereof. In some embodiments, the conjugate of compound 10 is R in FIG. 3A. In some embodiments, the antibody drug conjugate has the structure I:

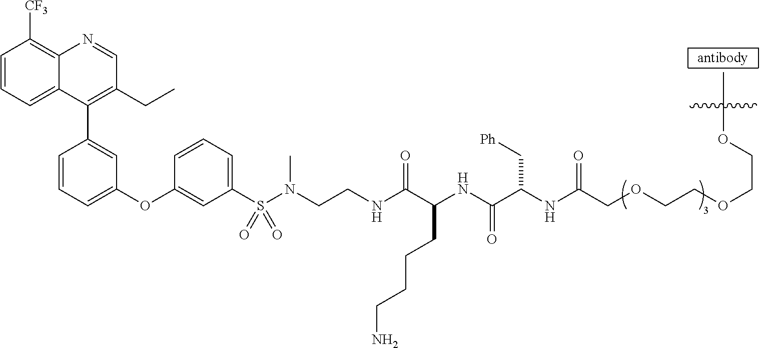

In another aspect, provided herein is a method for activating a liver X receptor in a leukocyte, the method comprising contacting the leukocyte with an antibody drug conjugate comprising (a) a liver X receptor agonist and (b) an antibody specific for a cell surface molecule of the leukocyte. In some embodiments, the cell surface molecule is not present on a hepatocyte. In some embodiments, the cell surface molecule is an integrin. In some embodiments, the integrin is CD11a. In some embodiments, the antibody has a heavy chain variable domain having at least about 80% sequence identity to SEQ ID NO. 12. In some embodiments, the antibody has a heavy chain variable domain that differs from SEQ ID NO. 12 by less than about 5 amino acid residues. In some embodiments, the antibody comprises a heavy chain CDR1 sequence identical to SEQ ID NO. 19. In some embodiments, the antibody comprises a heavy chain CDR1 protein sequence that differs from SEQ ID NO. 19 by 3, 2 or 1 amino acid residues. In some embodiments, the antibody comprises a heavy chain CDR2 protein sequence identical to SEQ ID NO. 20. In some embodiments, the antibody comprises a heavy chain CDR2 protein sequence that differs from SEQ ID NO. 20 by 3, 2 or 1 amino acid residues. In some embodiments, the antibody comprises a heavy chain CDR3 protein sequence identical to SEQ ID NO. 21. In some embodiments, the antibody comprises a heavy chain CDR3 protein sequence that differs from SEQ ID NO. 21 by 3, 2 or 1 amino acid residues. In some embodiments, the antibody comprises a light chain CDR1 protein sequence identical to SEQ ID NO. 22. In some embodiments, the antibody comprises a light chain CDR1 protein sequence that differs from SEQ ID NO. 22 by 3, 2 or 1 amino acid residues. In some embodiments, the antibody comprises a light chain CDR2 protein sequence identical to SEQ ID NO. 23. In some embodiments, the antibody comprises a light chain CDR2 protein sequence that differs from SEQ ID NO. 23 by 3, 2 or 1 amino acid residues. In some embodiments, the antibody comprises a light chain CDR3 protein sequence identical to SEQ ID NO. 24. In some embodiments, the antibody comprises a light chain CDR3 protein sequence that differs from SEQ ID NO. 24 by 3, 2 or 1 amino acid residues.

In some embodiments, the liver X receptor is liver X receptor alpha. In some embodiments, the liver X receptor is liver X receptor beta. In some embodiments, the antibody is chemically linked to (a) the liver X receptor agonist or (b) a moiety chemically attached to the liver X receptor agonist. In some embodiments, the antibody comprises an unnatural amino acid chemically linked to (a) the liver X receptor agonist or (b) the moiety chemically attached to the liver X receptor agonist. In some embodiments, the unnatural amino acid is para-acetylphenylalanine (pAcF). In some embodiments, the antibody has a heavy chain sequence having at least about 90% sequence identity to SEQ ID NO. 10. In some embodiments, the antibody has a heavy chain sequence that differs from SEQ ID NO. 10 by less than about 5 amino acid residues. In some embodiments, the antibody is chemically linked to the moiety chemically attached to the liver X receptor agonist. In some embodiments, the moiety comprises a protease cleavage site. In some embodiments, the liver X receptor agonist comprises compound 3 or a derivative or conjugate thereof. In some embodiments, the liver X receptor agonist comprises compound 10 or a derivative or conjugate thereof. In some embodiments, the conjugate of compound 10 is R in FIG. 3A. In some embodiments, the antibody drug conjugate has the structure I:

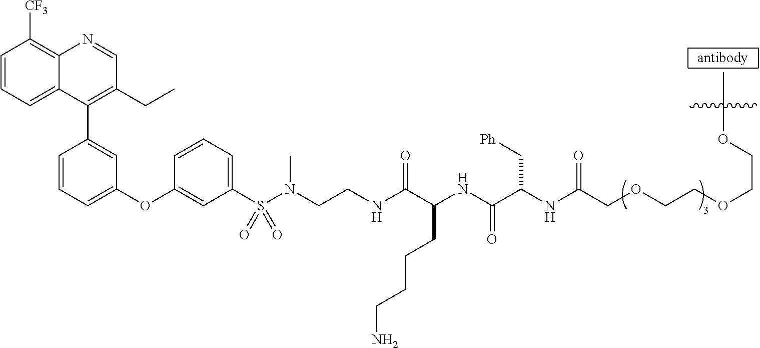

In another aspect, provided herein is a method for treating a subject in need thereof with a liver X receptor agonist, the method comprising administering to the subject an antibody drug conjugate comprising (a) the liver X receptor agonist, and (b) an antibody specific for an integrin expressed on a leukocyte. In some embodiments, the integrin is not expressed on a hepatocyte. In some embodiments, the subject has a disease or condition selected from atherosclerosis, diabetes, inflammation, and Alzheimer's disease. In some embodiments, the leukocyte is a macrophage. In some embodiments, the macrophage is a foam cell. In some embodiments, the integrin is selected from CD11a, CD11b, and CD11c. In some embodiments, the antibody has a heavy chain variable domain having at least about 80% sequence identity to SEQ ID NO. 12. In some embodiments, the antibody has a heavy chain variable domain that differs from SEQ ID NO. 12 by less than about 5 amino acid residues. In some embodiments, the antibody comprises a heavy chain CDR1 sequence identical to SEQ ID NO. 19. In some embodiments, the antibody comprises a heavy chain CDR1 protein sequence that differs from SEQ ID NO. 19 by 3, 2 or 1 amino acid residues. In some embodiments, the antibody comprises a heavy chain CDR2 protein sequence identical to SEQ ID NO. 20. In some embodiments, the antibody comprises a heavy chain CDR2 protein sequence that differs from SEQ ID NO. 20 by 3, 2 or 1 amino acid residues. In some embodiments, the antibody comprises a heavy chain CDR3 protein sequence identical to SEQ ID NO. 21. In some embodiments, the antibody comprises a heavy chain CDR3 protein sequence that differs from SEQ ID NO. 21 by 3, 2 or 1 amino acid residues. In some embodiments, the antibody comprises a light chain CDR1 protein sequence identical to SEQ ID NO. 22. In some embodiments, the antibody comprises a light chain CDR1 protein sequence that differs from SEQ ID NO. 22 by 3, 2 or 1 amino acid residues. In some embodiments, the antibody comprises a light chain CDR2 protein sequence identical to SEQ ID NO. 23. In some embodiments, the antibody comprises a light chain CDR2 protein sequence that differs from SEQ ID NO. 23 by 3, 2 or 1 amino acid residues. In some embodiments, the antibody comprises a light chain CDR3 protein sequence identical to SEQ ID NO. 24. In some embodiments, the antibody comprises a light chain CDR3 protein sequence that differs from SEQ ID NO. 24 by 3, 2 or 1 amino acid residues.

In some embodiments, the liver X receptor agonist comprises compound 3 or a derivative or conjugate thereof. In some embodiments, the liver X receptor agonist comprises compound 10 or a derivative or conjugate thereof. In some embodiments, the conjugate of compound 10 is depicted as R in FIG. 3A. In some embodiments, the liver X receptor agonist binds to liver X receptor alpha within the leukocyte. In some embodiments, the liver X receptor agonist binds to liver X receptor beta within the leukocyte. In some embodiments, the antibody is chemically linked to (a) the liver X receptor agonist or (b) a moiety chemically attached to the liver X receptor agonist. In some embodiments, the antibody comprises an unnatural amino acid chemically linked to (a) the liver X receptor agonist or (b) the moiety chemically attached to the liver X receptor agonist. In some embodiments, the unnatural amino acid is para-acetylphenylalanine (pAcF). In some embodiments, the antibody has a heavy chain sequence having at least about 90% sequence identity to SEQ ID NO. 10. In some embodiments, the antibody has a heavy chain sequence that differs from SEQ ID NO. 10 by less than about 5 amino acid residues. In some embodiments, the antibody is chemically linked to the moiety chemically attached to the liver X receptor agonist. In some embodiments, the moiety comprises a protease cleavage site. In some embodiments, the antibody drug conjugate has the structure I:

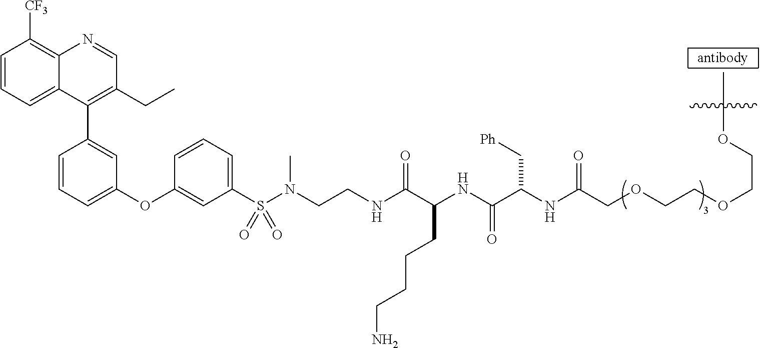

Further disclosed herein are antibody drug conjugates comprising: an antibody or antibody fragment that binds a cell surface molecule on a target cell, wherein the target cell is a non-malignant lymphocyte; and a therapeutic agent that has a therapeutic effect in a subject in need thereof. Further disclosed herein are antibody drug conjugates comprising: an antibody or antibody fragment that binds a cell surface molecule on a target cell; and a therapeutic agent that binds an intracellular moiety of the target cell. The target cell may be selected from a monocyte, macrophage, foam cell, T cell, platelet, endothelial cell, endothelial cell progenitor, and vascular smooth muscle cell. The macrophage may express an inflammatory marker. The inflammatory marker may be selected from CD64, CD68, CD80, CD86, CD11a, CD11b, CD11c and CD163. The inflammatory marker may be a cell surface molecule on the target cell. The macrophage may be an activated macrophage. The macrophage may be a cholesterol-laden macrophage. The macrophage may be present in an atherosclerotic plaque or atherosclerotic lesion. The cell surface molecule may internalize upon binding of the antibody drug conjugate to the cell surface molecule. The cell surface molecule may be an integrin. The integrin is may be selected from CD11a, CD11b, and CD11c. The integrin may be CD11a. The intracellular moiety may comprise a peptide. The intracellular moiety may be a receptor. The receptor may be a nuclear receptor. The therapeutic agent may comprise a moiety selected from a compound, a peptide, and a combination thereof. The moiety may comprise a ligand that interacts with an intracellular receptor of the target cell. The ligand may be a ligand for a nuclear receptor. The moiety may modulate a function of a nuclear receptor. The nuclear receptor may be selected from a liver X receptor alpha, a liver X receptor beta, a peroxisome-proliferator activated receptor alpha, a peroxisome-proliferator activated receptor delta, a peroxisome-proliferator activated receptor gamma, a farnesoid X receptor, a hepatocyte nuclear factor 4, a vitamin D receptor, a glucocorticoid receptor, a mineralocorticoid receptor and a progesterone receptor. The nuclear receptor may be selected from liver X receptor alpha and liver X receptor beta. The therapeutic agent may modulate a cellular function of the target cell, wherein the cellular function is selected from an inflammatory activity, lipid regulation, cholesterol regulation, apoptosis, migration, chemotaxis, gene transcription, and protein expression. The antibody or antibody fragment may bind an antigen selected from a human antigen, a simian antigen, and a murine antigen. The antibody or antibody fragment may be selected from an anti-CD64 antibody, anti-CD163 antibody, anti-CD86 antibody, an anti-CD80 antibody, an anti-CD11a antibody, an anti-CD11b antibody, an anti-CD11c antibody, fragments thereof and combinations thereof. The antibody or antibody fragment may be selected from an anti-CD11a antibody and a fragment thereof.

Disclosed herein are antibody drug conjugates comprising an anti-CD11a antibody or antibody fragment and a liver X receptor ligand. Further disclosed herein are antibody drug conjugate comprising a liver X receptor ligand and an antibody or antibody fragment selected from an anti-CD64 antibody, anti-CD163 antibody, anti-CD86 antibody, an anti-CD80 antibody, an anti-CD11a antibody, an anti-CD11b antibody, an anti-CD11c antibody, fragments thereof and combinations thereof. The liver X receptor ligand may be a liver X receptor agonist. The agonist may possess a chemical structure selected from a chemical structure depicted in FIG. 1, and similar derivatives thereof.

Disclosed herein are methods of treating a condition comprising administering to a subject in need thereof an antibody drug conjugate disclosed herein. The condition may be a cardiovascular condition. The condition may be a cardiovascular disease. The cardiovascular disease may be selected from atherosclerosis, ischemic heart disease (IHD), stroke, hypertensive heart disease, aortic aneurysm, endocarditis, and peripheral artery disease (PAD), and a combination thereof. The condition may not be cancer. The condition may not be an autoimmune disease.

Further disclosed herein are pharmaceutical compositions comprising: any one of the antibody drug conjugates disclosed herein, and a pharmaceutically acceptable salt, excipient or vehicle.

### BRIEF DESCRIPTION OF THE DRAWINGS

FIG. 1 shows exemplary synthetic LXR agonists and their LXR-α and LXR-β binding affinities, in a simplified binding model.

FIG. 2 shows an exemplary scheme for synthesis of an aminooxy-modified Cathepsin B-LXR agonist (compound 10).

FIG. 3A shows site-specific conjugation of a CatB-LXR agonist (compound 10) to anti-CD11a IgGX via a ketone-hydroxylamine condensation reaction in acetate buffer at 37° C. to form anti-CD11a IgGX-CatB-LXR agonist (molecule 20 comprising agonist R) with a stable oxime linkage.

FIG. 3B shows an SDS-PAGE analysis of antibodies prepared herein, reduced (lanes 5 and 6) or not reduced (lanes 2 and 3), with DTT treatment; Lanes 2 and 5 show anti-CD11a IgGX, Lanes 3 and 6 show anti-CD11a IgGX-CatB-LXR agonist, Lane 1 is a molecular weight marker.

FIG. 3C shows ESI-MS data showing both the light chain and CatB-LXR agonist-modified heavy chain of an anti-CD11a IgGX-CatB-LXR agonist after treatment with PNGase F (Promega, 12 h at 37° C. in PBS pH 7.4) and subsequent reduction of the disulfide bonds with 10 mM DTT: Calculated MW=50,699 Da, Observed MW=50,697 Da.

FIG. 4A is a flow cytometry histogram showing binding of AF488-conjugated antibodies on THP-1 cells. Cells were incubated either with 2 nM of the dye-conjugated antibodies or with increasing concentrations of antibody (0-50 nM, right) for 1 h at 4° C. in the absence of Fc block.

FIG. 4B is a flow cytometry histogram showing binding of AF-488-conjugated antibodies on THP-1 cells. Cells were incubated either with 2 nM of the dye-conjugated antibodies or with increasing concentrations of antibody (0-50 nM, right) for 1 h at 4° C. in the presence of Fc block (Human Fc block, BD Biosciences).

FIG. 4C is a flow cytometry histogram showing lack of binding of AF-488-conjugated antibodies to HepG2 cells.

FIG. 4D shows binding specificity of anti-CD11a IgGX-AF488 and anti-Her2 IgGX-AF488 on THP-1(+) Fc block cells as shown by a plot of mean fluorescence intensity (MFI) versus concentration of antibody.

FIG. 4E Confocal microscopy analysis of anti-CD11a IgGX internalization in the presence of Fc block. THP-1 cells were incubated with 50 nM anti-CD11a IgGX-AF488 for 2 h at I) 37° C., II) 4° C., or III) in the presence of 1 μM anti-CD11a IgGX at 37° C. Anti-Her2 IgGX-AF488 (IV) was used as a negative control. Cells were then fixed, stained with Hoechst (blue, nucleus), Alexa Fluor 594-conjugated wheat germ agglutinin (red, membrane) and imaged with a Zeiss 710 confocal microscope. Bar=10 μm.

FIG. 5A shows an evaluation of agonist activity of anti-CD11a IgGX-CatB-LXR agonist, T0901317 small molecule agonist (2), and aminooxy-CatB-LXR agonist free drug (10) in the presence and absence of 10% human AB serum on THP-1 cells. Data were normalized to the highest concentration of T0901317 (set at 100%).

FIG. 5B shows an evaluation of agonist activity of anti-CD11a IgGX-CatB-LXR agonist, T0901317 small molecule agonist (2), and aminooxy-CatB-LXR agonist free drug (10) in the presence and absence of 10% human AB serum on HepG2 cells. Data were normalized to the highest concentration of T0901317 (set at 100%).

FIGS. 6(A-F) show dose-response curves from the LanthaScreen binding assays of different synthetic LXR agonists. Emission ratio of 520 nm to 490 nm was calculated first and normalized in Genedata. EC₅₀ (AC₅₀ from the graph) was calculated based on the fitted curve.

FIG. 6A shows the binding assay for LXR-α agonist 1.

FIG. 6B shows the binding assay for LXR-α agonist 2.

FIG. 6C shows the binding assay for LXR-α agonist 3.

FIG. 6D shows the binding assay for LXR-β agonist 1.

FIG. 6E shows the binding assay for LXR-β agonist 2.

FIG. 6F shows the binding assay for LXR-β agonist 3.

FIG. 7A is a plot of percent compound remaining over time in a CatB-LXR agonist cleavage assay. Capped parent compound 10 was incubated either in THP-1 cell growth media or in the presence of Cathepsin B at 37° C. over a period of 24 h. After the indicated times, samples were extracted and the loss of the parent compound was analyzed by LC-MS and plotted, as shown in FIG. 7A.

FIG. 7B shows capped parent compound 10 and cleaved product 3 after CatB cleavage.

FIG. 8A is an ESI-MS spectra of anti-CD11a pAcF IgGX. Light chain expected MW=23412 Da, observed MW=23409 Da. Heavy chain expected MW=49664 Da, observed MW=49663 Da.

FIG. 8B is an ESI-MS spectra of anti-Her2 IgGX. Light chain expected MW=23443 Da, observed MW=23440 Da. Heavy chain expected MW=49241 Da, observed MW=49239 Da.

FIG. 8C is an ESI-MS spectra of anti-Her2 FabX. Expected MW=47812 Da, observed MW=47819 Da. anti-CD11a IgGX and anti-Her2 IgGX were treated with PNGase F (Promega, 12 h at 37° C. in PBS pH 7.4) to remove N-glycans and 10 mM DTT to afford reduced light chains (LC) and heavy chains (HC).

FIG. 9A is a FACS histogram analysis of binding of anti-CD11a IgGX-AF488 to THP-1 cells at different concentrations (0, 0.08, 0.4, 2, 10, and 50 nM), with or without Fc block treatment.

FIG. 9B is a FACS histogram analysis of binding of anti-Her2 to THP-1 cells at different concentrations (0, 0.08, 0.4, 2, 10, and 50 nM), with or without Fc block treatment.

FIG. 9C shows mean relative fluorescence intensities of the binding of AF488-conjugated antibodies to THP-1 cells in the presence or absence of Fc block.

FIG. 10A shows FACS analysis of the binding of different Alexa Fluor 488-antibody conjugates (2 nM) on HepG2 cells without Fc block treatment.

FIG. 10B shows FACS analysis of the binding of different AF488-conjugated antibodies to THP-1 cells with Fc block treatment.

FIG. 11 shows internalization of anti-CD11a IgGX labeled with Alexa Fluor 488 analyzed by confocal microscopy. THP-1 cells were treated with 50 nM of anti-CD11a IgGX-AF488 or anti-Her2 IgGX-AF488 in the presence or absence of 1 μM of unlabeled anti-CD11a IgGX at 37° C. (or 4° C.) for 2 h, with or without Fc block. Only anti-CD11a IgGX-AF488-treated cells showed internalization even after Fey receptors were blocked. Bar=10 μm.

FIG. 12 shows a chemical synthesis scheme for the production of compound 14.

FIG. 13 shows a chemical synthesis scheme for the production of compound 9.

FIG. 14 shows a chemical synthesis scheme for the production of compound 10.

FIG. 15A is an ESI-MS spectra of anti-CD11a IgGX-AF488. Samples in FIG. were treated with PNGase F (Promega, 12 h at 37° C. in PBS pH 7.4) to remove N-glycans and 10 mM DTT to afford reduced light chains (LC) and heavy chains (HC).

FIG. 15B is an ESI-MS spectra of anti-Her2 IgGX-AF488. Samples were treated with PNGase F (Promega, 12 h at 37° C. in PBS pH 7.4) to remove N-glycans and 10 mM DTT to afford reduced light chains (LC) and heavy chains (HC).

FIG. 15C is an ESI-MS spectra of anti-Her2 FabX-AF488.

### DETAILED DESCRIPTION OF THE INVENTION

Atherosclerosis is a chronic progressive disease that accounts for approximately 35% of all deaths in the United States. One of the underlying processes that drives atherosclerosis is the formation of oxidized low-density lipoproteins (oxLDL) and a subsequent inflammatory response. Macrophages recruited as part of this response engulf, but fail to adequately process, oxLDL particles and transform into lipid-laden foam cells whose accumulation in the intima leads to the formation of atherosclerotic lesions. When left untreated, these lesions will cause the vessel to rupture leading to fatal thrombosis. Under non-pathogenic conditions, normal macrophages exert atheroprotective effects by promoting the efflux of cholesterol to the liver for bile secretion via reverse cholesterol transport (RCT). RCT is triggered upon activation of the liver X receptors (LXR-α and LXR-β), sterol-responsive nuclear receptors that control the transcription of several genes directly involved in cholesterol homeostasis and lipid metabolism, such as ATP-binding cassette transporters ABCG1 and ABCA1, lipoprotein lipase (LPL), and apolipoprotein E (ApoE). Additionally, LXR activation suppresses an array of inflammatory genes, namely, iNOS, COX-2, IL-6, and MCP-1/3 and results in inhibition of macrophage proliferation and foam cell formation.

While both LXR-α and LXR-β can be found in monocytes and macrophages as well as other tissues (intestine, kidney, adipose), LXR-α is also highly expressed in the liver. In contrast to LXR activation in macrophages, activation of LXR in the liver increases hepatic triglyceride levels, which is an independent risk factor for the development of atherosclerosis. Indeed, although numerous studies demonstrated significant reduction in atherosclerotic lesions in murine models upon treatment with synthetic LXR agonists GW3965 and T0901317, a significant increase in hepatic lipogenesis and plasma triglyceride levels was also observed. This effect on triglyceride levels was notably absent in LXR-α knockout mice treated with synthetic LXR agonist, giving rise to drug discovery efforts aimed at generating LXR-β-selective agonists. However, to date these efforts have not delivered compounds that have progressed into patients, likely due to an inability to obtain sufficient selectivity. Targeted delivery of an LXR agonist to macrophages by an ADC represents a fundamentally new approach to selectively engage the LXR pathway in the treatment of atherosclerosis.

Disclosed herein are novel site-specific antibody-drug conjugates (ADCs) that selectively deliver an LXR agonist to monocytes/macrophages while sparing hepatocytes. The unnatural amino acid para-acetylphenylalanine (pAcF) is site-specifically incorporated into anti-CD11a IgG, which binds the α-chain component of the lymphocyte function-associated antigen 1 (LFA-1) expressed on nearly all monocytes and macrophages. An aminooxy-modified LXR agonist is conjugated to the anti-CD11a IgG through a stable, cathepsin B cleavable oxime linkage to afford a chemically-defined ADC. This novel ADC represents a fundamentally different strategy that uses tissue targeting to overcome the limitations of LXR agonists for potential use in treating atherosclerosis, namely avoiding unwanted lipogenic effects in hepatocytes.

### I. Antibody Drug Conjugates

Disclosed herein are antibody drug conjugates comprising: an antibody or antibody fragment that binds a cell surface molecule on a target cell, wherein the target cell is a non-malignant lymphocyte; and a therapeutic agent that has a therapeutic effect in a subject in need thereof. As used herein, the term “non-malignant lymphocyte” refers to a blood cell that is not associated with, derived from or causative of a cancer or otherwise cellular proliferative disease or disorder. In this way, the antibody drug conjugates disclosed herein are distinguished from antibody drug conjugates designed to treat cancers, malignancies or tumors.

Further disclosed herein are antibody drug conjugates comprising: an antibody or antibody fragment that binds a cell surface molecule on a target cell; and a therapeutic agent that binds an intracellular moiety of the target cell. As used herein, the term “intracellular moiety” refers to a moiety that resides within the cell. The intracellular moiety may be in contact with or integrated with the cellular membrane. However, the region of the intracellular moiety that is bound by the therapeutic agent is not exposed to the exterior or outside of the cell. In some cases, no part of the intracellular moiety is exposed to the exterior or outside of the cell.

Before the present methods, kits and compositions are described in greater detail, it is to be understood that this invention is not limited to particular method, kit or composition described, as such may, of course, vary. It is also to be understood that the terminology used herein is for the purpose of describing particular embodiments only, and is not intended to be limiting, since the scope of the present invention will be limited only by the appended claims. Examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

It must be noted that as used herein and in the appended claims, the singular forms “a”, “an”, and “the” include plural referents unless the context clearly dictates otherwise. Thus, for example, reference to “a cell” includes a plurality of such cells and reference to “the peptide” includes reference to one or more peptides and equivalents thereof, e.g. polypeptides, known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### A. Antibodies and Antibody Fragments

Disclosed herein are antibody drug conjugates comprising antibodies or antibody fragments. As used herein, the term “antibody fragment” refers to any form of an antibody other than the full-length form. Antibody fragments herein include antibodies that are smaller components that exist within full-length antibodies, and antibodies that have been engineered. Antibody fragments include, but are not limited to, Fv, Fc, Fab, and (Fab′)2, single chain Fv (scFv), diabodies, triabodies, tetrabodies, bifunctional hybrid antibodies, CDR1, CDR2, CDR3, combinations of CDRs, variable regions, framework regions, constant regions, heavy chains, light chains, alternative scaffold non-antibody molecules, and bispecific antibodies. Unless specifically noted otherwise, statements and claims that use the term “antibody” or “antibodies” may specifically include “antibody fragment” and “antibody fragments.”

The targeting polypeptide may be human, fully human, humanized, human engineered, non-human, and/or chimeric antibody. The non-human antibody may be humanized to reduce immunogenicity to humans, while retaining the specificity and affinity of the parental non-human antibody. Generally, a humanized antibody comprises one or more variable domains in which CDRs (or portions thereof) are derived from a non-human antibody, and FRs (or portions thereof) are derived from human antibody sequences. A humanized antibody optionally also comprises at least a portion of a human constant region. In some embodiments, some FR residues in a humanized antibody are substituted with corresponding residues from a non-human antibody (e.g., the antibody from which the CDR residues are derived), e.g., to restore or improve antibody specificity or affinity.

Chimeric antibodies may refer to antibodies created through the joining of two or more antibody genes which originally encoded for separate antibodies. A chimeric antibody may comprise at least one amino acid from a first antibody and at least one amino acid from a second antibody, wherein the first and second antibodies are different. At least a portion of the antibody or antibody fragment may be from a bovine species, a human species, or a murine species. At least a portion of the antibody or antibody fragment may be from a rat, a goat, a guinea pig or a rabbit. At least a portion of the antibody or antibody fragment may be from a human. At least a portion of the antibody or antibody fragment may be from cynomolgus monkey.

The antibody or antibody fragment may target an antigen on the target cell, wherein the target cell is present at/in an atherosclerotic plaque/lesion. The antibody or antibody fragment may target a cell surface molecule on a cell that influences the state/progression of an atherosclerotic plaque/lesion.

The antibody or antibody fragment may be selected from an anti-CD64 antibody, an anti-CD163 antibody, an anti-CD86 antibody, an anti-CD80 antibody, an anti-CD11a antibody, an anti-CD11b antibody, an anti-CD11c antibody, an anti-CD14 antibody, an anti-CD36 antibody, an anti-scavenger receptor A antibody, an anti-LDLR antibody, an anti-TLR2 antibody, an anti-TLR4 antibody, an anti-CXCR4 antibody, an anti-IL-6R antibody, an anti-IL-18R antibody, an anti-TNF-receptor antibody, an anti-CD40 antibody, an anti-interferon receptor antibody, an anti-TGFβR antibody, an anti-CCR2 antibody, an anti-CCR4 antibody, an anti-CD68 antibody, and fragments thereof, and combinations thereof.

In some embodiments, the antibody comprises a DNA sequence of any of SEQ ID NOS. 1-9. In some embodiments, the antibody comprises a DNA sequence at least about 70%, 80%, 90%, 95%, or 99% identical to any of SEQ ID NOS. 1-9. In some embodiments, the antibody comprises a DNA sequence that differs from any of SEQ ID NOS. 1-9 by less than about 20, 15, 10, 9, 8, 7, 6, 5, 4, 3, or 2 bases. In some embodiments, the antibody comprises a heavy chain variable domain DNA sequence at least about 70%, 80%, 90%, 95%, or 99% identical to SEQ ID NO. 3. In some embodiments, the antibody comprises a heavy chain variable domain DNA sequence of SEQ ID NO. 3. In some embodiments, the antibody comprises a heavy chain variable domain DNA sequence that differs from SEQ ID NO. 3 by less than about 20, 15, 10, 9, 8, 7, 6, 5, 4, 3, or 2 bases. In some embodiments, the antibody comprises a light chain variable domain DNA sequence at least about 70%, 80%, 90%, 95%, or 99% identical to SEQ ID NO. 8. In some embodiments, the antibody comprises a light chain variable domain DNA sequence of SEQ ID NO. 8. In some embodiments, the antibody comprises a light chain variable domain DNA sequence that differs from SEQ ID NO. 8 by less than about 20, 15, 10, 9, 8, 7, 6, 5, 4, 3, or 2 bases.

In some embodiments, the antibody comprises a protein sequence of any of SEQ ID NOS. 10-18. In some embodiments, the antibody comprises a protein sequence at least about 70%, 80%, 90%, 95%, or 99% identical to any of SEQ ID NOS. 10-18. In some embodiments, the antibody comprises a protein sequence that differs from any of SEQ ID NOS. 10-18 by less than about 20, 15, 10, 9, 8, 7, 6, 5, 4, 3, or 2 amino acids. In some embodiments, the antibody comprises a heavy chain variable domain protein sequence at least about 70%, 80%, 90%, 95%, or 99% identical to SEQ ID NO. 12. In some embodiments, the antibody comprises a heavy chain variable domain protein sequence of SEQ ID NO. 12. In some embodiments, the antibody comprises a heavy chain variable domain protein sequence that differs from SEQ ID NO. 12 by less than about 20, 15, 10, 9, 8, 7, 6, 5, 4, 3, or 2 amino acids. In some embodiments, the antibody comprises a light chain variable domain protein sequence at least about 70%, 80%, 90%, 95%, or 99% identical to SEQ ID NO. 17. In some embodiments, the antibody comprises a light chain variable domain protein sequence of SEQ ID NO. 17. In some embodiments, the antibody comprises a light chain variable domain protein sequence that differs from SEQ ID NO. 17 by less than about 20, 15, 10, 9, 8, 7, 6, 5, 4, 3, or 2 amino acids.

In some embodiments, the antibody comprises a heavy chain CDR1 protein sequence identical to SEQ ID NO. 19. In some embodiments, the antibody comprises a heavy chain CDR1 protein sequence that differs from SEQ ID NO. 19 by 3, 2 or 1 amino acids. In some embodiments, the antibody comprises a heavy chain CDR2 protein sequence identical to SEQ ID NO. 20. In some embodiments, the antibody comprises a heavy chain CDR2 protein sequence that differs from SEQ ID NO. 20 by 3, 2 or 1 amino acids. In some embodiments, the antibody comprises a heavy chain CDR3 protein sequence identical to SEQ ID NO. 21. In some embodiments, the antibody comprises a heavy chain CDR3 protein sequence that differs from SEQ ID NO. 21 by 3, 2 or 1 amino acids. In some embodiments, the antibody comprises a light chain CDR1 protein sequence identical to SEQ ID NO. 22. In some embodiments, the antibody comprises a light chain CDR1 protein sequence that differs from SEQ ID NO. 22 by 3, 2 or 1 amino acids. In some embodiments, the antibody comprises a light chain CDR2 protein sequence identical to SEQ ID NO. 23. In some embodiments, the antibody comprises a light chain CDR2 protein sequence that differs from SEQ ID NO. 23 by 3, 2 or 1 amino acids. In some embodiments, the antibody comprises a light chain CDR3 protein sequence identical to SEQ ID NO. 24. In some embodiments, the antibody comprises a light chain CDR3 protein sequence that differs from SEQ ID NO. 24 by 3, 2 or 1 amino acids.

In some embodiments, the antibody comprises a modified Fc region. In some embodiments, the Fc region is modified to reduce antibody-dependent cellular cytotoxicity (ADCC) and complement-dependent cytotoxicity (CDC). For example, the Fc region of an antibody provided herein, including the anti-CD11a antibodies of Table 1, has one or more of the following mutations: E233P, L234V, L235A, deltaG236, A327G, A330S, and P331S. In some embodiments, the Fc region is modified with L238A and/or L239A mutations. For example, SEQ ID NO. 10 is modified with L238A and L239A.

The antibody or antibody fragment may bind an integrin. The integrin may be selected from CD49a, CD49b, CD49c, CD49d, CD49e, CD49f, ITGA7, ITGA8, ITGA9, ITGA10, ITGA11, CD11a, CD11b, CD11c, CD11d, CD103, CD51, CD41, CD29, CD18, CD61, CD104, ITGB5, ITGB6, ITGB7 and ITGB8. The integrin may be CD11a.

The antibody or antibody fragment may bind an antigen on the target cell, wherein the antigen is or is part of a cell surface molecule that internalizes upon binding of the antibody drug conjugate to the cell surface molecule, thereby allowing the therapeutic agent of the ADC to access an intracellular moiety/target. The antigen may be selected from a human antigen, a simian antigen, and a murine antigen.

### B. Therapeutic Agents

Disclosed herein are antibody drug conjugates comprising therapeutic agents. Therefore the antibody drug conjugates disclosed herein may also be referred to as “antibody-therapeutic agent conjugates.” These terms are used interchangeably herein.

The therapeutic agent may be selected from a protein, a peptide, a small molecule, and a compound. The therapeutic agent may bind an intracellular moiety. The intracellular moiety may be selected from a signaling molecule, a signaling peptide/protein, an enzyme, a scaffold protein, a non-peptide moiety, a receptor, and a combination thereof. The intracellular moiety may be a receptor. The receptor may be a nuclear hormone receptor (“nuclear receptor”). The nuclear receptor may be selected from a liver X receptor alpha, a liver X receptor beta, a peroxisome-proliferator activated receptor alpha, a peroxisome-proliferator activated receptor delta, a peroxisome-proliferator activated receptor gamma, a farnesoid X receptor, a hepatocyte nuclear factor 4, a vitamin D receptor, a glucocorticoid receptor, a mineralocorticoid receptor and a progesterone receptor. One skilled in the art would see how additional nuclear receptors would be targeted with a similar ADC containing a therapeutic agent that is a ligand for one or more of these additional nuclear receptors. Additional nuclear receptors include, but are not limited to, thyroid hormone receptors, retinoic acid receptors, Rev-ErbA, RAR-related orphan receptors, constitutive androstane receptor, retinoid x receptors, estrogen receptors, estrogen-related receptors and androgen receptor.

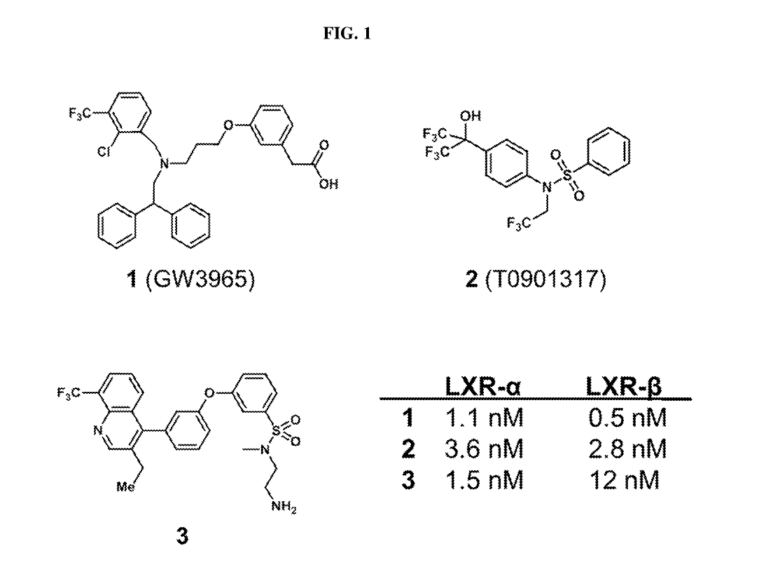

The therapeutic agent may comprise a ligand that interacts with an intracellular receptor of the target cell. The therapeutic agent may comprise a ligand for a nuclear receptor. The ligand for the nuclear receptor may an LXR agonist. The LXR agonist may be selected from hypocholamide, T0901317, GW3965, and N,N-dimethyl-3beta-hydroxy-cholenamide (DMHCA). The ligand may be selected from a chemical structure depicted in FIG. 1, or a similar derivative thereof. Similar derivatives thereof may be those chemical structures depicted in FIG. 1 modified with chemical groups or moieties that do not substantially change the ligand's LXR-mediated activity or effects. By “not substantially change,” it is meant that the ligand's LXR-mediated activity or effects do not differ by more than about 10% from the ligand's LXR-mediated activity, wherein the ligand possesses a chemical structure depicted in FIG. 1, as measured by an LXR activity/function such as, but not limited to, LXR-mediated transcription and LXR-mediated cholesterol efflux.

The ligand for the nuclear receptor may a PPAR agonist. The PPAR agonist may be selected from a PPAR alpha agonist, a PPAR delta agonist and a PPAR gamma agonist. The PPAR agonist may be selected from a fibrates, a thizolidinediones, an NSAID, a glitizar, clofibrate, gemfibrozil, ciprofibrate, bezafibrate, fenofibrate, GW501516, aleglitazar, muraglitazar, tesaglitazar, rosiglitazone, pioglitazone, lobeglitazone, troglitazone, netoglitazone, rivoglitazone, and ciglitazone.

The therapeutic agent may interact with cellular proteins or molecules other than nuclear receptors as well. For example, the therapeutic agent may be selected from a number of agents used to treat cardiovascular disease, including, by way of non-limiting example, an HMG-CoA reductase inhibitor (e.g. statins), an ACE inhibitor, an aldosterone inhibitor, an angiotensin receptor (e.g. angiotensin II receptor) blocker, a calcium channel blocker, a cholesterol-lowering drug, digoxin, a diuretic, a vasodilator, an anti-coagulant (e.g. warfarin), and an anti-inflammatory agent (e.g. NSAID, flavonoid).

The therapeutic agent may modulate a function of a nuclear receptor. By way of non-limiting example, the function may be selected from expression/secretion of inflammatory mediators (e.g. cytokines, chemokines), cholesterol regulation, cholesterol intake, cholesterol efflux, cholesterol oxidation, migration, chemotaxis, apoptosis and necrosis, an inflammatory activity, lipid regulation, apoptosis, migration, chemotaxis, gene transcription, and protein expression.

### C. Linkers and Conjugation Methods

Disclosed herein are antibody drug conjugates comprising an antibody or antibody fragment and a therapeutic agent. Generally, the antibody or antibody fragment and the therapeutic agent are site-specifically conjugated. Compared to non-specific conjugation that utilizes surface-exposed lysines on the antibody, site-specific conjugation strategies have been shown to improve stability, pharmacokinetics, and the drug safety profile of the resulting ADCs. The antibody or antibody fragment may comprise an unnatural amino acid, wherein the antibody or antibody fragment and the therapeutic agent are linked/conjugated via the unnatural amino acid. In some cases, the therapeutic agent comprises a peptide and the unnatural amino acid is located in the peptide.

The unnatural amino acid may be inserted between two naturally occurring amino acids in the antibody or antibody fragment. The one or more unnatural amino acids may replace one or more naturally occurring amino acids in the antibody or antibody fragment. The one or more unnatural amino acids may be incorporated at the N terminus of the antibody or antibody fragment. The one or more unnatural amino acids may be incorporated at the C terminus of the antibody or antibody fragment. The unnatural amino acid may be incorporated distal to the binding region of antibody or antibody fragment. The unnatural amino acid may be incorporated near the binding region of the antibody or antibody fragment. The unnatural amino acid may be incorporated in the binding region of the antibody or antibody fragment.

The one or more unnatural amino acids may be encoded by a codon that does not code for one of the twenty natural amino acids. The one or more unnatural amino acids may be encoded by a nonsense codon (stop codon). The stop codon may be an amber codon. The amber codon may comprise a UAG sequence. The stop codon may be an ochre codon. The ochre codon may comprise a UAA sequence. The stop codon may be an opal or umber codon. The opal or umber codon may comprise a UGA sequence. The one or more unnatural amino acids may be encoded by a four-base codon.

The one or more unnatural amino acids may be p-acetylphenylalanine (pAcF or pAcPhe). The one or more unnatural amino acids may be selenocysteine. The one or more unnatural amino acids may be p-fluorophenylalanine (pFPhe). The one or more unnatural amino acids may be selected from the group comprising p-azidophenylalanine (pAzF), p-azidomethylphenylalanine (pAzCH₂F), p-benzoylphenylalanine (pBpF), p-propargyloxyphenylalanine (pPrF), p-iodophenylalanine (pIF), p-cyanophenylalanine (pCNF), p-carboxylmethylphenylalanine (pCmF), 3-(2-naphthyl)alanine (NapA), p-boronophenylalanine (pBoF), o-nitrophenylalanine (oNiF), (8-hydroxyquinolin-3-yl)alanine (HQA), selenocysteine, and (2,2′-bipyridin-5-yl)alanine (BipyA).). The one or more unnatural amino acids may be 4-(6-methyl-s-tetrazin-3-yl)aminopheynlalanine.

The one or more unnatural amino acids may be β-amino acids (03 and (32), homo-amino acids, proline and pyruvic acid derivatives, 3-substituted alanine derivatives, glycine derivatives, ring-substituted phenylalanine and tyrosine derivatives, linear core amino acids, diamino acids, D-amino acids, N-methyl amino acids, or a combination thereof.

Additional examples of unnatural amino acids include, but are not limited to, 1) various substituted tyrosine and phenylalanine analogues such as O-methyl-L-tyrosine, p-amino-L-phenylalanine, 3-nitro-L-tyrosine, p-nitro-L-phenylalanine, m-methoxy-L-phenylalanine and p-isopropyl-L-phenylalanine; 2) amino acids with aryl azide and benzophenone groups that may be photo-cross-linked; 3) amino acids that have unique chemical reactivity including acetyl-L-phenylalanine and m-acetyl-L-phenylalanine, O-allyl-L-tyrosine, O-(2-propynyl)-L-tyrosine, p-ethylthiocarbonyl-L-phenylalanine and p-(3-oxobutanoyl)-L-phenylalanine; 4) heavy-atom-containing amino acids for phasing in X-ray crystallography including p-iodo and p-bromo-L-phenylalanine; 5) the redox-active amino acid dihydroxy-L-phenylalanine; 6) glycosylated amino acids including b-N-acetylglucosamine-O-serine and a-N-acetylgalactosamine-O-threonine; 7) fluorescent amino acids with naphthyl, dansyl, and 7-aminocoumarin side chains; 8) photocleavable and photoisomerizable amino acids with azobenzene and nitrobenzyl Cys, Ser, and Tyr side chains; 9) the phosphotyrosine mimetic p-carboxymethyl-L-phenylalanine; 10) the glutamine homologue homoglutamine; and 11) 2-aminooctanoic acid. The unnatural amino acid may be modified to incorporate a chemical group. The unnatural amino acid may be modified to incorporate a ketone group.

The one or more unnatural amino acids may comprise at least one oxime, carbonyl, dicarbonyl, hydroxylamine group or a combination thereof. The one or more unnatural amino acids may comprise at least one carbonyl, dicarbonyl, alkoxy-amine, hydrazine, acyclic alkene, acyclic alkyne, cyclooctyne, aryl/alkyl azide, norbornene, cyclopropene, trans-cyclooctene, or tetrazine functional group or a combination thereof.

The one or more unnatural amino acids may be incorporated into the antibody or antibody fragment by methods known in the art. Cell-based or cell-free systems may be used to alter the genetic sequence of antibody or antibody fragment, thereby producing the antibody or antibody fragment with one or more unnatural amino acids. Auxotrophic strains may be used in place of engineered tRNA and synthetase. The one or more unnatural amino acids may be produced through selective reaction of one or more natural amino acids. The selective reaction may be mediated by one or more enzymes. In one non-limiting example, the selective reaction of one or more cysteines with formylglycine generating enzyme (FGE) may produce one or more formylglycines as described in Rabuka et al., Nature Protocols 7:1052-1067 (2012).

The one or more unnatural amino acids may take part in a chemical reaction to form a linker. The chemical reaction to form the linker may be a bioorthogonal reaction. The chemical reaction to form the linker may be click chemistry.

Additional unnatural amino acids are disclosed in Liu et al. (Annu Rev Biochem, 79:413-44, 2010), Wang et al. (Angew Chem Int Ed, 44:34-66, 2005) and PCT application numbers PCT/US2012/039472, PCT/US2012/039468, PCT/US2007/088009, PCT/US2009/058668, PCT/US2007/089142, PCT/US2007/088011, PCT/US2007/001485, PCT/US2006/049397, PCT/US2006/047822 and PCT/US2006/044682, all of which are incorporated by reference in their entireties.

The one or more unnatural amino acids may replace one or more amino acids in the antibody or antibody fragment. The one or more unnatural amino acids may replace any natural amino acid in the antibody or antibody fragment.

The one or more unnatural amino acids may be incorporated in a light chain of the antibody or antibody fragment. The one or more unnatural amino acids may be incorporated in a heavy chain of the antibody or antibody fragment. The one or more unnatural amino acids may be incorporated in a heavy chain and a light chain of antibody or antibody fragment. The one or more unnatural amino acids may replace an amino acid in the light chain of the antibody or antibody fragment. The one or more unnatural amino acids may replace an amino acid in a heavy chain of the antibody or antibody fragment. The one or more unnatural amino acids may replace an amino acid in a heavy chain and a light chain of the antibody or antibody fragment.

The one or more unnatural amino acids may replace an amino acid of the antibody or antibody fragment, wherein the antibody or antibody fragment is an anti-CD11a antibody or fragment thereof.

The antibody drug conjugates disclosed herein may comprise a linker that links that antibody or antibody fragment and the therapeutic agent. The antibody drug conjugate may be produced by conjugating a first portion of the linker to the antibody or antibody fragment and a second portion of the linker to the therapeutic agent. Conjugating the linker to the antibody or antibody fragment or the therapeutic agent may comprise production of an ionic bond, a covalent bond, a non-covalent bond or a combination thereof between the linker and the antibody, antibody fragment or therapeutic agent. Conjugating the linker to the antibody or antibody fragment or the therapeutic agent may be performed as described in Roberts et al., Advanced Drug Delivery Reviews 54:459-476 (2002). The linker may be selected from a bifunctional linker, a cleavable linker, a non-cleavable linker, an ethylene glycol linker, a bifunctional ethylene glycol linker, a flexible linker, or an inflexible linker. The linker may comprise a chemical group selected from a cyclooctyne, a cyclopropene, an aryl/alkyl azide, a trans-cyclooctene, a norborene, and a tetrazine. A terminus of the linker comprise an alkoxy-amine. A terminus of the linker comprise an azide or cyclooctyne group. The antibody or antibody fragment or therapeutic agent may be coupled to the linker by a chemical group selected from a cyclooctyne, cyclopropene, aryl/alkyl azide, trans-cyclooctene, norborene, and tetrazine. Linking the antibody or antibody fragment or therapeutic agent to the linker may comprise conducting one or more copper-free reactions. Linking the antibody or antibody fragment or therapeutic agent to the linker may comprise conducting one or more copper-containing reactions. Linking the antibody or antibody fragment or therapeutic agent to the linker may comprise one or more cycloadditions. Linking the antibody or antibody fragment or therapeutic agent to the linker may comprise one or more Huisgen-cycloadditions. Linking the antibody or antibody fragment or therapeutic agent to the linker may comprise one or more Diels Alder reactions. Linking the antibody or antibody fragment or therapeutic agent to the linker may comprise one or more Hetero Diels Alder reaction.

### D. Target Cells

Disclosed herein are antibody drug conjugates comprising: an antibody or antibody fragment that binds a cell surface molecule on a target cell. The target cell may be a monocyte. The target cell may be a macrophage. The target cell may be a phagocytic macrophage. The target cell may be a cholesterol-laden macrophage. The target cell may be a foam cell (e.g. laden with oxidized LDL). The target cell may be an activated macrophage. The macrophage may be present in an atherosclerotic plaque or atherosclerotic lesion. The target cell may be in a pro-inflammatory state. The target cell may be in an anti-inflammatory state. The cell may be an endothelial cell. The cell may be a vascular smooth muscle cell. The cell may be a platelet.

The target cell may be a non-malignant lymphocyte. As used herein, a non-malignant lymphocyte refers to a lymphocyte that is not associated with or derived from a cancer or tumor. Thus, the methods and compositions disclosed herein target non-malignant lymphocyte and do not aim to treat cancer, tumors, or malignancies.

The target cell may express an inflammatory marker. The inflammatory marker may be selected from a C-reactive protein, serum amyloid A, fibrinogen, an adhesion molecule (e.g. ICAM, PECAM, VCAM), a matrix metalloprotease (MMP), an interleukin, a toll-like receptor, an interferon, a tumor necrosis factor, receptors thereof, ligands thereof, and combinations thereof. The inflammatory marker may be the cell surface molecule on the target cell.

### II. Pharmaceutical Compositions

Disclosed herein is a pharmaceutical composition comprising one or more of the antibody drug conjugates disclosed herein. The compositions may further comprise one or more pharmaceutically acceptable salts, excipients or vehicles. Pharmaceutically acceptable salts, excipients, or vehicles for use in the present pharmaceutical compositions include carriers, excipients, diluents, antioxidants, preservatives, coloring, flavoring and diluting agents, emulsifying agents, suspending agents, solvents, fillers, bulking agents, buffers, delivery vehicles, tonicity agents, cosolvents, wetting agents, complexing agents, buffering agents, antimicrobials, and surfactants.

Neutral buffered saline or saline mixed with serum albumin are exemplary appropriate carriers. The pharmaceutical compositions may include antioxidants such as ascorbic acid; low molecular weight polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as Tween, pluronics, or polyethylene glycol (PEG). Also by way of example, suitable tonicity enhancing agents include alkali metal halides (preferably sodium or potassium chloride), mannitol, sorbitol, and the like. Suitable preservatives include benzalkonium chloride, thimerosal, phenethyl alcohol, methylparaben, propylparaben, chlorhexidine, sorbic acid and the like. Hydrogen peroxide also may be used as preservative. Suitable cosolvents include glycerin, propylene glycol, and PEG. Suitable complexing agents include caffeine, polyvinylpyrrolidone, beta-cyclodextrin or hydroxy-propyl-beta-cyclodextrin. Suitable surfactants or wetting agents include sorbitan esters, polysorbates such as polysorbate 80, tromethamine, lecithin, cholesterol, tyloxapal, and the like. The buffers may be conventional buffers such as acetate, borate, citrate, phosphate, bicarbonate, or Tris-HCl. Acetate buffer may be about pH 4-5.5, and Tris buffer may be about pH 7-8.5. Additional pharmaceutical agents are set forth in Remington's Pharmaceutical Sciences, 18th Edition, A. R. Gennaro, ed., Mack Publishing Company, 1990.

The composition may be in liquid form or in a lyophilized or freeze-dried form and may include one or more lyoprotectants, excipients, surfactants, high molecular weight structural additives and/or bulking agents (see, for example, U.S. Pat. Nos. 6,685,940, 6,566,329, and 6,372,716). In one embodiment, a lyoprotectant is included, which is a non-reducing sugar such as sucrose, lactose or trehalose. The amount of lyoprotectant generally included is such that, upon reconstitution, the resulting formulation will be isotonic, although hypertonic or slightly hypotonic formulations also may be suitable. In addition, the amount of lyoprotectant should be sufficient to prevent an unacceptable amount of degradation and/or aggregation of the protein upon lyophilization. Exemplary lyoprotectant concentrations for sugars (e.g., sucrose, lactose, trehalose) in the pre-lyophilized formulation are from about 10 mM to about 400 mM. In another embodiment, a surfactant is included, such as for example, nonionic surfactants and ionic surfactants such as polysorbates (e.g., polysorbate 20, polysorbate 80); poloxamers (e.g., poloxamer 188); poly(ethylene glycol) phenyl ethers (e.g., Triton); sodium dodecyl sulfate (SDS); sodium laurel sulfate; sodium octyl glycoside; lauryl-, myristyl-, linoleyl-, or stearyl-sulfobetaine; lauryl-, myristyl-, linoleyl- or stearyl-sarcosine; linoleyl, myristyl-, or cetyl-betaine; lauroamidopropyl-, cocamidopropyl-, linoleamidopropyl-, myristamidopropyl-, palmidopropyl-, or isostearamidopropyl-betaine (e.g., lauroamidopropyl); myristamidopropyl-, palmidopropyl-, or isostearamidopropyl-dimethylamine; sodium methyl cocoyl-, or disodium methyl ofeyl-taurate; and the MONAQUAT™. series (Mona Industries, Inc., Paterson, N.J.), polyethyl glycol, polypropyl glycol, and copolymers of ethylene and propylene glycol (e.g., Pluronics, PF68 etc). Exemplary amounts of surfactant that may be present in the pre-lyophilized formulation are from about 0.001-0.5%. High molecular weight structural additives (e.g., fillers, binders) may include for example, acacia, albumin, alginic acid, calcium phosphate (dibasic), cellulose, carboxymethylcellulose, carboxymethylcellulose sodium, hydroxyethylcellulose, hydroxypropylcellulose, hydroxypropylmethylcellulose, microcrystalline cellulose, dextran, dextrin, dextrates, sucrose, tylose, pregelatinized starch, calcium sulfate, amylose, glycine, bentonite, maltose, sorbitol, ethylcellulose, disodium hydrogen phosphate, disodium phosphate, disodium pyrosulfite, polyvinyl alcohol, gelatin, glucose, guar gum, liquid glucose, compressible sugar, magnesium aluminum silicate, maltodextrin, polyethylene oxide, polymethacrylates, povidone, sodium alginate, tragacanth microcrystalline cellulose, starch, and zein. Exemplary concentrations of high molecular weight structural additives are from 0.1% to 10% by weight. In other embodiments, a bulking agent (e.g., mannitol, glycine) may be included.

Compositions may be suitable for parenteral administration. Exemplary compositions are suitable for injection or infusion into an animal by any route available to the skilled worker, such as intraarticular, subcutaneous, intravenous, intramuscular, intraperitoneal, intracerebral (intraparenchymal), intracerebroventricular, intramuscular, intraocular, intraarterial, or intralesional routes. A parenteral formulation typically will be a sterile, pyrogen-free, isotonic aqueous solution, optionally containing pharmaceutically acceptable preservatives.

Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringers' dextrose, dextrose and sodium chloride, lactated Ringer's, or fixed oils. Intravenous vehicles include fluid and nutrient replenishers, electrolyte replenishers, such as those based on Ringer's dextrose, and the like. Preservatives and other additives may also be present, such as, for example, anti-microbials, anti-oxidants, chelating agents, inert gases and the like. See generally, Remington's Pharmaceutical Science, 16th Ed., Mack Eds., 1980.

Pharmaceutical compositions described herein may be formulated for controlled or sustained delivery in a manner that provides local concentration of the product (e.g., bolus, depot effect) and/or increased stability or half-life in a particular local environment. The compositions may comprise the formulation of antibody drug conjugates disclosed herein with particulate preparations of polymeric compounds such as polylactic acid, polyglycolic acid, etc., as well as agents such as a biodegradable matrix, injectable microspheres, microcapsular particles, microcapsules, bioerodible particles beads, liposomes, and implantable delivery devices that provide for the controlled or sustained release of the active agent which then may be delivered as a depot injection. Techniques for formulating such sustained- or controlled-delivery means are known and a variety of polymers have been developed and used for the controlled release and delivery of drugs. Such polymers are typically biodegradable and biocompatible. Polymer hydrogels, including those formed by complexation of enantiomeric polymer or polypeptide segments, and hydrogels with temperature or pH sensitive properties, may be desirable for providing drug depot effect because of the mild and aqueous conditions involved in trapping bioactive protein agents (e.g., antibodies comprising an ultralong CDR3). See, for example, the description of controlled release porous polymeric microparticles for the delivery of pharmaceutical compositions in WO 93/15722. Suitable materials for this purpose include polylactides (see, e.g., U.S. Pat. No. 3,773,919), polymers of poly-(a-hydroxycarboxylic acids), such as poly-D-(−)-3-hydroxybutyric acid (EP 133,988A), copolymers of L-glutamic acid and gamma ethyl-L-glutamate (Sidman et al., Biopolymers, 22: 547-556 (1983)), poly(2-hydroxyethyl-methacrylate) (Langer et al., J. Biomed. Mater. Res., 15: 167-277 (1981), and Langer, Chem. Tech., 12: 98-105 (1982)), ethylene vinyl acetate, or poly-D(−)-3-hydroxybutyric acid. Other biodegradable polymers include poly(lactones), poly(acetals), poly(orthoesters), and poly(orthocarbonates). Sustained-release compositions also may include liposomes, which may be prepared by any of several methods known in the art (see, e.g., Eppstein et al., Proc. Natl. Acad. Sci. USA, 82: 3688-92 (1985)). The carrier itself, or its degradation products, should be nontoxic in the target tissue and should not further aggravate the condition. This may be determined by routine screening in animal models of the target disorder or, if such models are unavailable, in normal animals. Microencapsulation of recombinant proteins for sustained release has been performed successfully with human growth hormone (rhGH), interferon-(rhIFN-), interleukin-2, and MN rgp120. Johnson et al., Nat. Med., 2:795-799 (1996); Yasuda, Biomed. Ther., 27:1221-1223 (1993); Hora et al., Bio/Technology. 8:755-758 (1990); Cleland, “Design and Production of Single Immunization Vaccines Using Polylactide Polyglycolide Microsphere Systems,” in Vaccine Design: The Subunit and Adjuvant Approach, Powell and Newman, eds, (Plenum Press: New York, 1995), pp. 439-462; WO 97/03692, WO 96/40072, WO 96/07399; and U.S. Pat. No. 5,654,010. The sustained-release formulations of these proteins were developed using poly-lactic-coglycolic acid (PLGA) polymer due to its biocompatibility and wide range of biodegradable properties. The degradation products of PLGA, lactic and glycolic acids may be cleared quickly within the human body. Moreover, the degradability of this polymer may be depending on its molecular weight and composition. Lewis, “Controlled release of bioactive agents from lactide/glycolide polymer,” in: M. Chasin and R. Langer (Eds.), Biodegradable Polymers as Drug Delivery Systems (Marcel Dekker: New York, 1990), pp. 1-41. Additional examples of sustained release compositions include, for example, EP 58,481A, U.S. Pat. No. 3,887,699, EP 158,277A, Canadian Patent No. 1176565, U. Sidman et al., Biopolymers 22, 547 [1983], R. Langer et al., Chem. Tech. 12, 98 [1982], Sinha et al., J. Control. Release 90, 261 [2003], Zhu et al., Nat. Biotechnol. 18, 24 [2000], and Dai et al., Colloids Surf B Biointerfaces 41, 117 [2005].

Bioadhesive polymers are also contemplated for use in or with compositions of the present disclosure. Bioadhesives are synthetic and naturally occurring materials able to adhere to biological substrates for extended time periods. For example, Carbopol and polycarbophil are both synthetic cross-linked derivatives of poly(acrylic acid). Bioadhesive delivery systems based on naturally occurring substances include for example hyaluronic acid, also known as hyaluronan. Hyaluronic acid is a naturally occurring mucopolysaccharide consisting of residues of D-glucuronic and N-acetyl-D-glucosamine. Hyaluronic acid is found in the extracellular tissue matrix of vertebrates, including in connective tissues, as well as in synovial fluid and in the vitreous and aqueous humor of the eye. Esterified derivatives of hyaluronic acid have been used to produce microspheres for use in delivery that are biocompatible and biodegradable (see, for example, Cortivo et al., Biomaterials (1991) 12:727-730; EP 517,565; WO 96/29998; Illum et al., J. Controlled Rel. (1994) 29:133-141).

Both biodegradable and non-biodegradable polymeric matrices may be used to deliver compositions of the present disclosure, and such polymeric matrices may comprise natural or synthetic polymers. Biodegradable matrices are preferred. The period of time over which release occurs is based on selection of the polymer. Typically, release over a period ranging from between a few hours and three to twelve months is most desirable. Exemplary synthetic polymers which may be used to form the biodegradable delivery system include: polymers of lactic acid and glycolic acid, polyamides, polycarbonates, polyalkylenes, polyalkylene glycols, polyalkylene oxides, polyalkylene terepthalates, polyvinyl alcohols, polyvinyl ethers, polyvinyl esters, poly-vinyl halides, polyvinylpyrrolidone, polyglycolides, polysiloxanes, polyanhydrides, polyurethanes and co-polymers thereof, poly(butic acid), poly(valeric acid), alkyl cellulose, hydroxyalkyl celluloses, cellulose ethers, cellulose esters, nitro celluloses, polymers of acrylic and methacrylic esters, methyl cellulose, ethyl cellulose, hydroxypropyl cellulose, hydroxypropyl methyl cellulose, hydroxybutyl methyl cellulose, cellulose acetate, cellulose propionate, cellulose acetate butyrate, cellulose acetate phthalate, carboxylethyl cellulose, cellulose triacetate, cellulose sulphate sodium salt, poly(methyl methacrylate), poly(ethyl methacrylate), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate), polyethylene, polypropylene, poly(ethylene glycol), poly(ethylene oxide), poly(ethylene terephthalate), poly(vinyl alcohols), polyvinyl acetate, poly vinyl chloride, polystyrene and polyvinylpyrrolidone. Exemplary natural polymers include alginate and other polysaccharides including dextran and cellulose, collagen, chemical derivatives thereof (substitutions, additions of chemical groups, for example, alkyl, alkylene, hydroxylations, oxidations, and other modifications routinely made by those skilled in the art), albumin and other hydrophilic proteins, zein and other prolamines and hydrophobic proteins, copolymers and mixtures thereof. In general, these materials degrade either by enzymatic hydrolysis or exposure to water in vivo, by surface or bulk erosion. The polymer optionally is in the form of a hydrogel (see, for example, WO 04/009664, WO 05/087201, Sawhney, et al., Macromolecules, 1993, 26, 581-587) that can absorb up to about 90% of its weight in water and further, optionally is cross-linked with multi-valent ions or other polymers.

Delivery systems also include non-polymer systems that are lipids including sterols such as cholesterol, cholesterol esters and fatty acids or neutral fats such as mono-di- and tri-glycerides; hydrogel release systems; silastic systems; peptide based systems; wax coatings; compressed tablets using conventional binders and excipients; partially fused implants; and the like. Specific examples include, but are not limited to: (a) erosional systems in which the product is contained in a form within a matrix such as those described in U.S. Pat. Nos. 4,452,775, 4,675,189 and 5,736,152 and (b) diffusional systems in which a product permeates at a controlled rate from a polymer such as described in U.S. Pat. Nos. 3,854,480, 5,133,974 and 5,407,686. Liposomes containing the product may be prepared by methods known methods, such as for example (DE 3,218,121; Epstein et al., Proc. Natl. Acad. Sci. USA, 82: 3688-3692 (1985); Hwang et al., Proc. Natl. Acad. Sci. USA, 77: 4030-4034 (1980); EP 52,322; EP 36,676; EP 88,046; EP 143,949; EP 142,641; JP 83-118008; U.S. Pat. Nos. 4,485,045 and 4,544,545; and EP 102,324).

Alternatively or additionally, the compositions may be administered locally via implantation into the affected area of a membrane, sponge, or other appropriate material on to which an antibody drug conjugate disclosed herein has been absorbed or encapsulated. Where an implantation device is used, the device may be implanted into any suitable tissue or organ, and delivery of an antibody drug conjugate disclosed herein may be directly through the device via bolus, or via continuous administration, or via catheter using continuous infusion.

A pharmaceutical composition comprising an antibody drug conjugate disclosed herein may be formulated for inhalation, such as for example, as a dry powder. Inhalation solutions also may be formulated in a liquefied propellant for aerosol delivery. In yet another formulation, solutions may be nebulized. Additional pharmaceutical composition for pulmonary administration include, those described, for example, in WO 94/20069, which discloses pulmonary delivery of chemically modified proteins. For pulmonary delivery, the particle size should be suitable for delivery to the distal lung. For example, the particle size may be from 1 μm to 5 μm; however, larger particles may be used, for example, if each particle is fairly porous.

Certain formulations containing an antibody drug conjugate disclosed herein may be administered orally. Formulations administered in this fashion may be formulated with or without those carriers customarily used in the compounding of solid dosage forms such as tablets and capsules. For example, a capsule may be designed to release the active portion of the formulation at the point in the gastrointestinal tract when bioavailability is maximized and pre-systemic degradation is minimized. Additional agents may be included to facilitate absorption of a selective binding agent. Diluents, flavorings, low melting point waxes, vegetable oils, lubricants, suspending agents, tablet disintegrating agents, and binders also may be employed.

Another preparation may involve an effective quantity of an antibody drug conjugate disclosed herein in a mixture with non-toxic excipients which are suitable for the manufacture of tablets. By dissolving the tablets in sterile water, or another appropriate vehicle, solutions may be prepared in unit dose form. Suitable excipients include, but are not limited to, inert diluents, such as calcium carbonate, sodium carbonate or bicarbonate, lactose, or calcium phosphate; or binding agents, such as starch, gelatin, or acacia; or lubricating agents such as magnesium stearate, stearic acid, or talc.

Suitable and/or preferred pharmaceutical formulations may be determined in view of the present disclosure and general knowledge of formulation technology, depending upon the intended route of administration, delivery format, and desired dosage. Regardless of the manner of administration, an effective dose may be calculated according to patient body weight, body surface area, or organ size. Further refinement of the calculations for determining the appropriate dosage for treatment involving each of the formulations described herein are routinely made in the art and is within the ambit of tasks routinely performed in the art. Appropriate dosages may be ascertained through use of appropriate dose-response data.

### III. Methods of Treatment

Disclosed herein are methods of treating a condition comprising administering to a subject in need thereof an antibody drug conjugate disclosed herein. The condition may be a cardiovascular disease. The cardiovascular disease may be selected from atherosclerosis, ischemic heart disease (IHD), stroke, hypertensive heart disease, aortic aneurysm, endocarditis, and peripheral artery disease (PAD), and a combination thereof. The condition may not be cancer. The condition may not be an autoimmune disease.

### EXAMPLES

The following illustrative examples are representative of embodiments of the software applications, systems, and methods described herein and are not meant to be limiting in any way.

### Example 1

### Design and Synthesis of the LXR Agonist

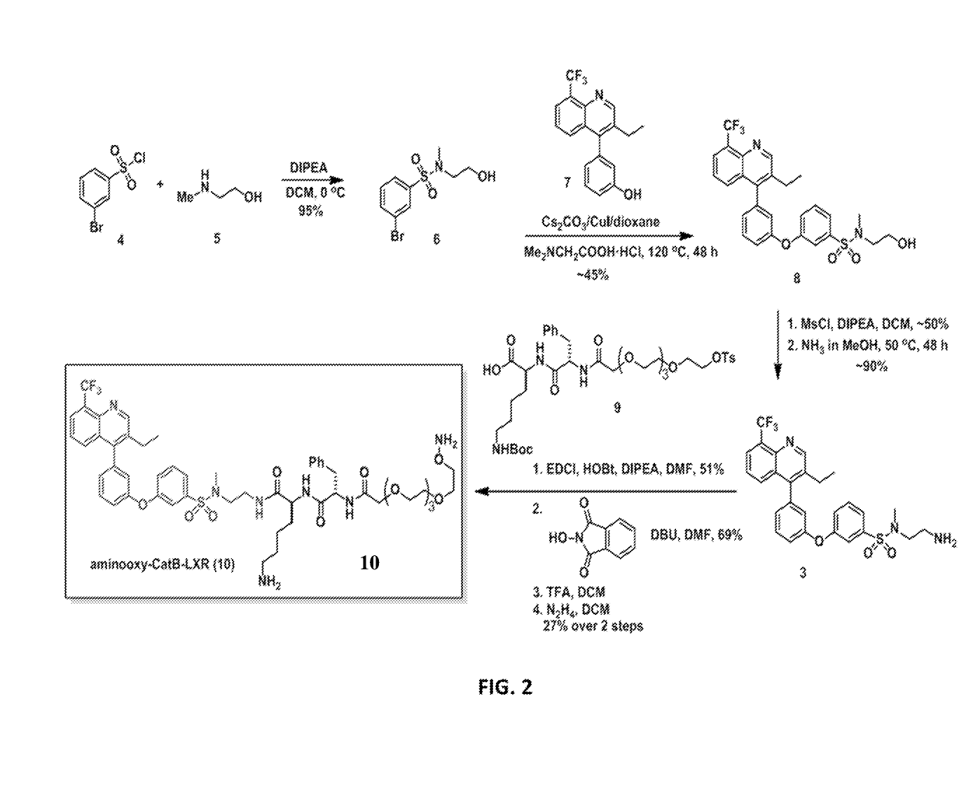

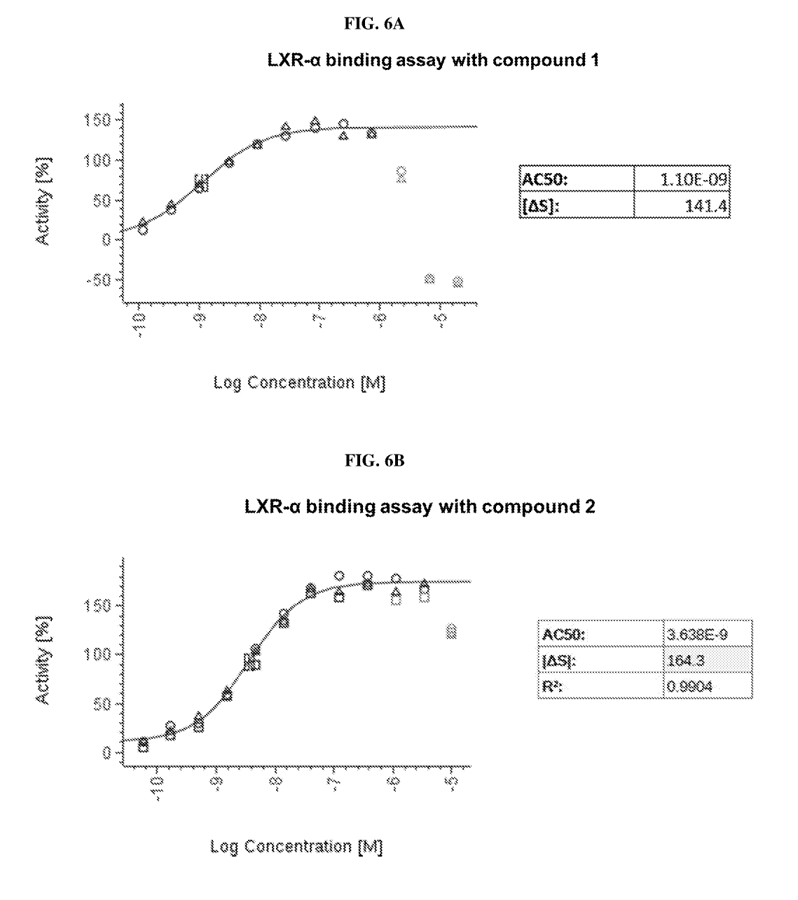

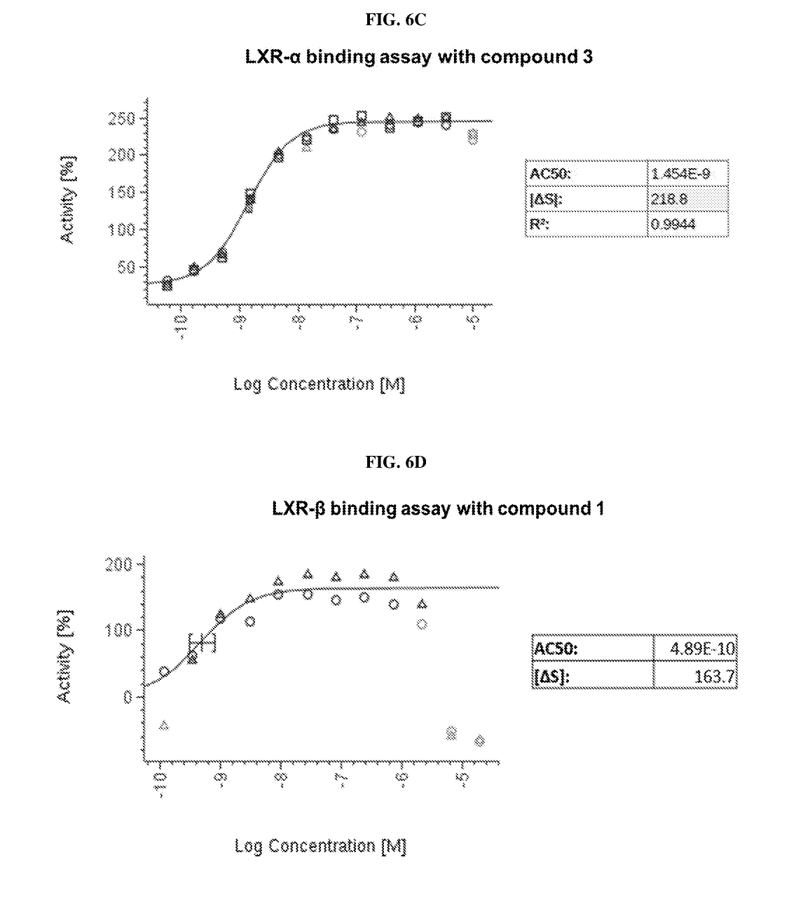

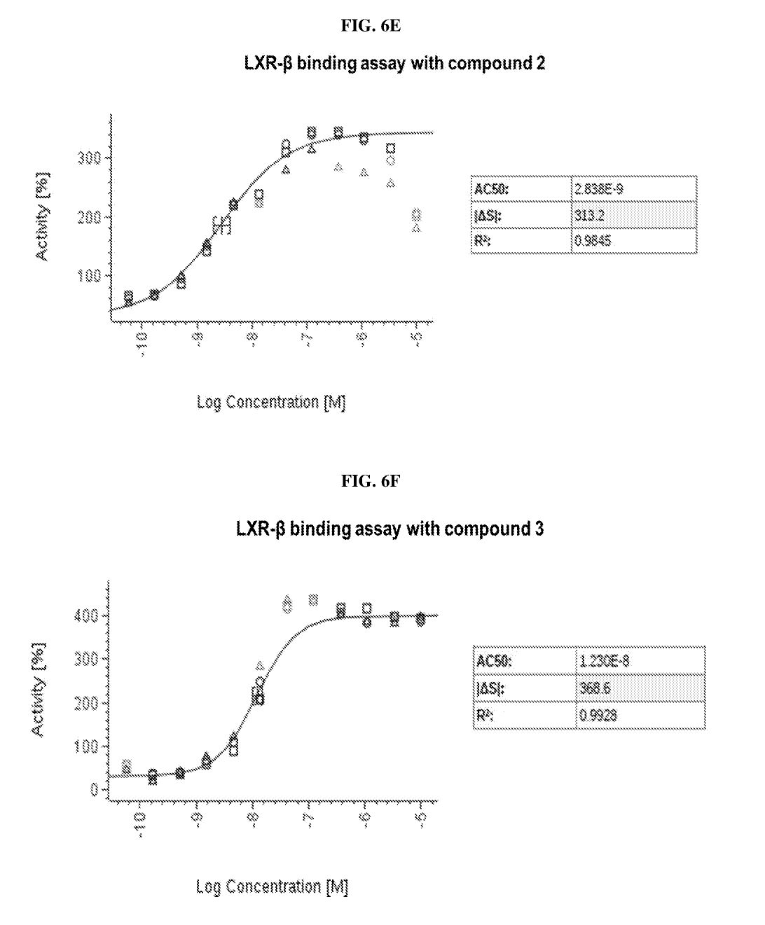

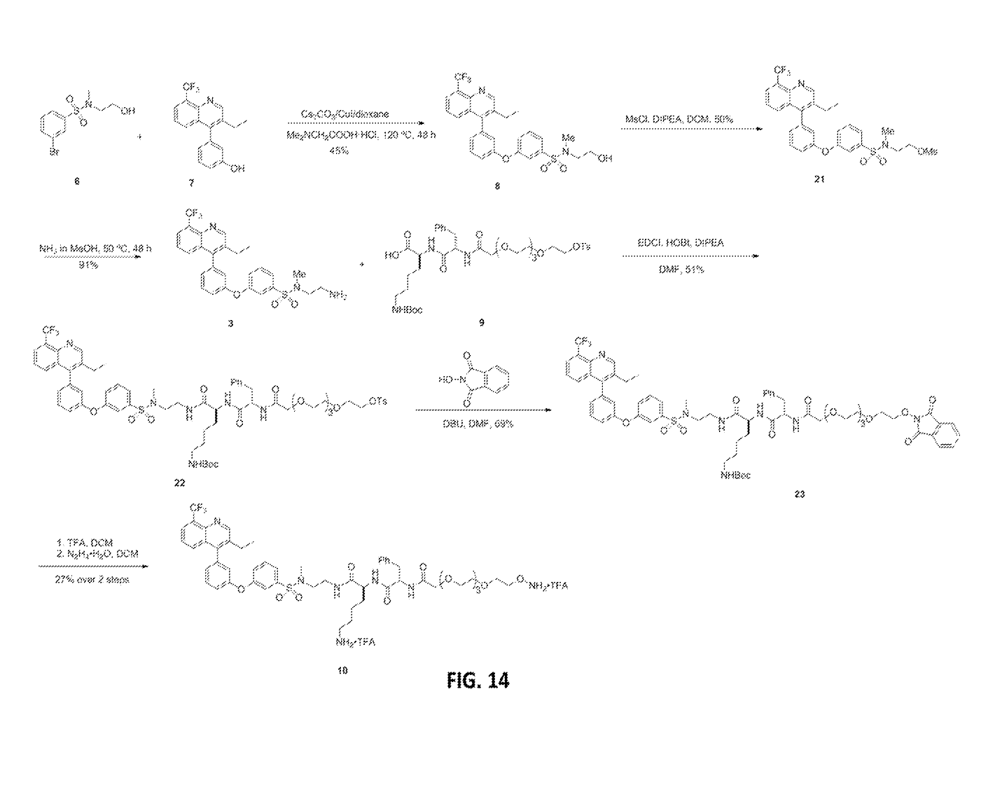

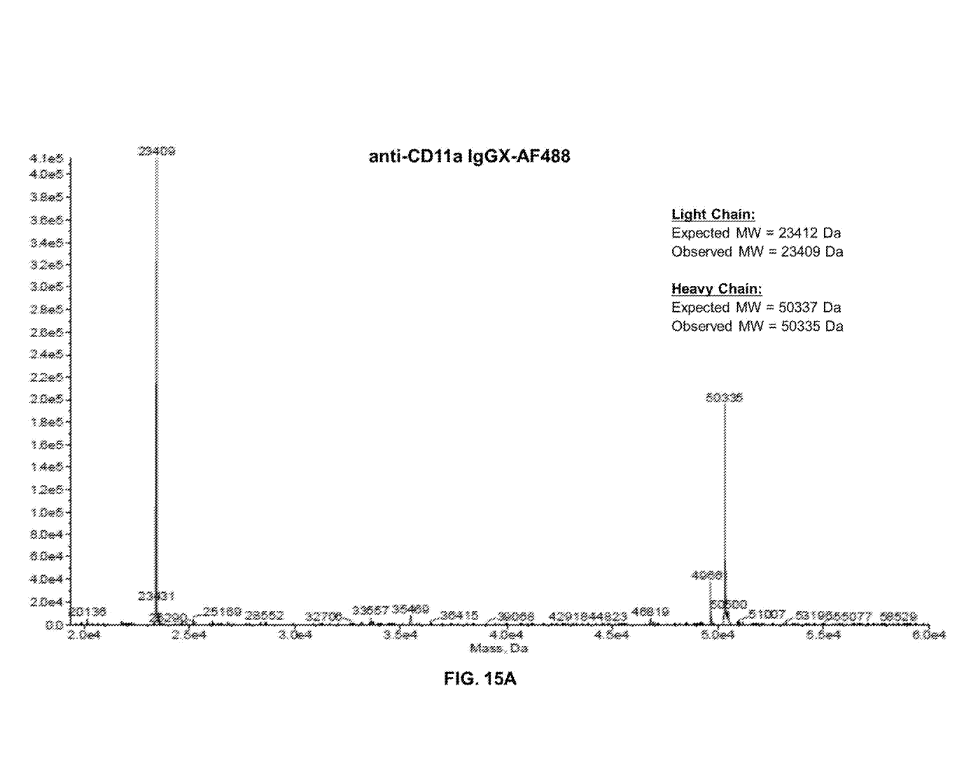

GW396523 and T09013178, shown in FIG. 1, are two widely used LXR agonists to study cardiovascular diseases. However, based on the reported structure-activity relationships (SAR), these two agonists fit tightly within the LXR receptor binding site and, as such, would be difficult to derivatize with a linker for antibody conjugation while retaining potency. A highly potent LXR agonist (compound 8, FIG. 2) was identified that tolerates modification at its sulfonamide group. To determine whether the core structures of these agonists can be used to generate highly potent, antibody-linked LXR agonists, compound 3 was synthesized from compound 8 with an aminoethyl sulfonamide substituent as shown in the schemes of FIG. 2 and FIG. 14. LXR binding with compound 3 was tested in comparison with compound 1 and compound 2 (LXR-α/LXR-β LanthaScreen, Life Technologies). Indeed, the modification of compound 8 is well-tolerated and compound 3 exhibits similar binding affinity for the LXR receptor binding site as GW3965 and T0901317. As shown in FIG. 1 and FIGS. 6(A-F), the binding affinity of compound 3 is 1.5 nM for LXR-α, and 12 nM for LXR-β. In comparison, the binding affinity of compound 1 is 1.1 nM for LXR-α, and 0.5 nM for LXR-β. The binding affinity of compound 2 is 3.6 nM for LXR-α, and 2.8 nM for LXR-β.

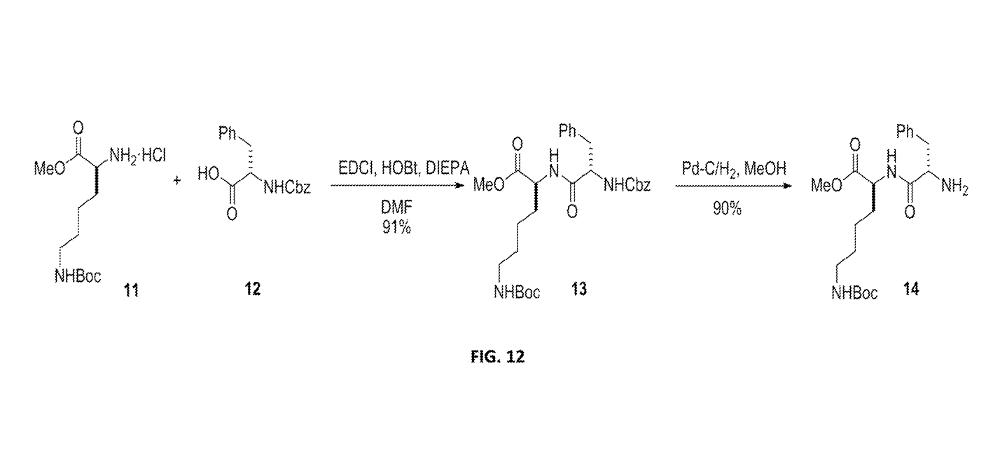

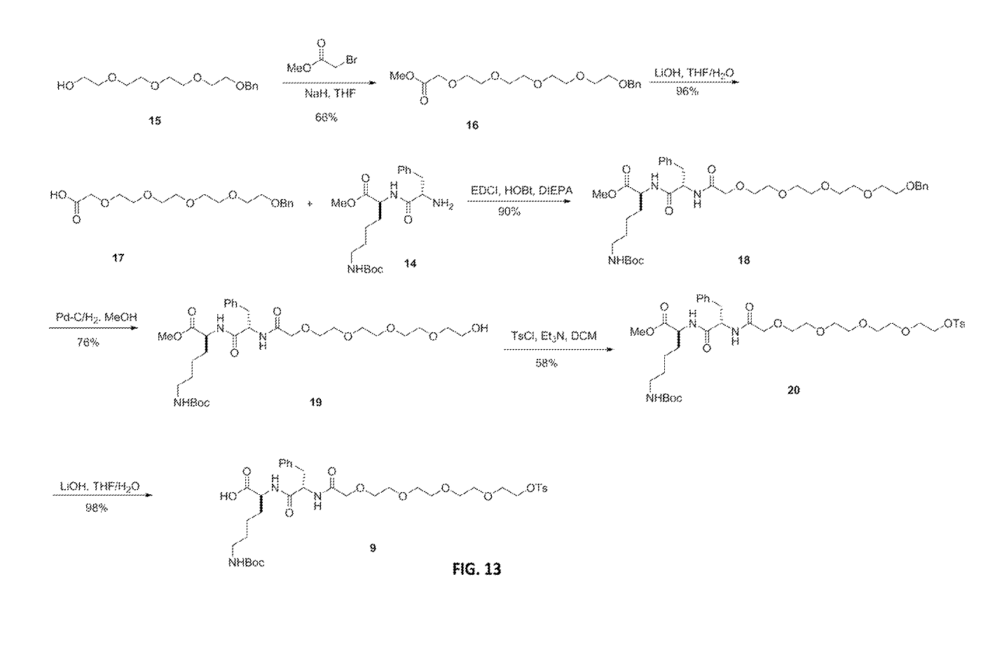

Next, compound 3 was designed and synthesized with a linker for use in antibody conjugation. Several different linker release strategies were considered, including disulfides, acid-labile hydrazones, and protease cleavable linkers. Among these, a protease cleavable phenylalanine-lysine (Phe-Lys) dipeptide, a stable linker that is rapidly hydrolyzed by the lysosomal enzyme Cathepsin B (CatB), was chosen resulting in the release of the free LXR agonist compound 3 inside the cell. A terminal aminooxy moiety was also incorporated to allow for site-specific conjugation to the antibody. To synthesize aminooxy-modified CatB-LXR agonist compound 10, 3-bromobenzenesulphonyl chloride (compound 4) was reacted with 2-(methylamino)ethanol (compound 5) to afford 3-bromobenzenesulfonamide (compound 6) in 95% yield (Scheme 1, FIG. 2). Next, compound 6 was coupled to a commercially-available quinolone (compound 7) in the presence of dimethylglycine hydrochloride in a cesium carbonate/copper(I) iodide/dioxane solution to afford compound 8 in moderate yield. The alcohol group of compound 8 was then converted to the methanesulfonate, which in turn was converted to amine to generate compound 3 in high yield using ammonia in methanol. Coupling of compound 3 with the pre-formed tosylated-PEGylated dipeptide (compound 9) (generated using the schemes of FIG. 12 and FIG. 13) was carried out using EDCI/HOBt in 51% yield. The resulting product was reacted with N-hydroxyphthalimide to form the alkoxyamine. Sequential deprotection of the Boc and phthalimide groups provided the final product, aminooxy-CatB-LXR agonist (compound 10) in an overall 27% yield (Scheme 1, FIG. 2, FIG. 14).

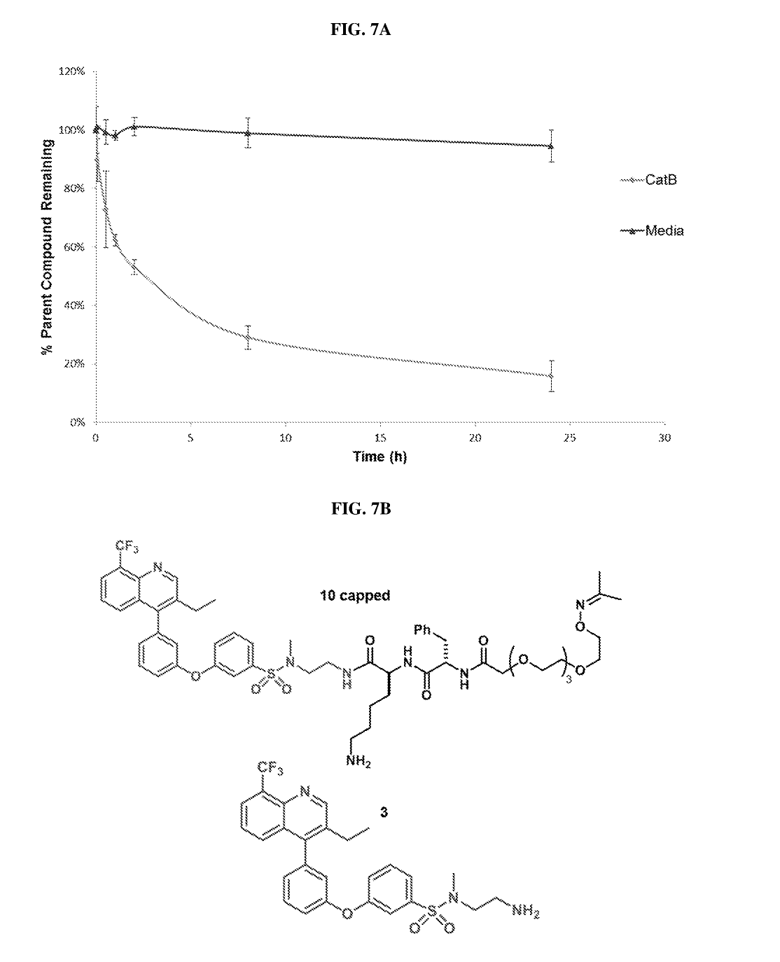

The stability of aminooxy-CatB-LXR agonist was next evaluated in cell culture media. A capped compound 10 (FIG. 7B) was incubated in growth media (RPMI, 10% FBS, 0.1% β-mercaptoethanol, 1 mM sodium pyruvate and 100 U/ml penicillin-streptomycin) at 37° C. Samples were extracted at different time points and release of parent compound was analyzed by LC-MS. The results indicate the aminooxy-CatB-LXR agonist is completely stable after 24 hours. (FIG. 7A). Cleavage of the Phe-Lys dipeptide was also analyzed by incubating capped compound 10 with purified CatB enzyme (EMD Millipore). Notably, after 2 hours of incubation with CatB, formation of a new peak was observed that corresponds to the mass of the desired cleavage product, compound 3 (FIG. 7B). Taken together, these results indicate the aminooxy-CatB-LXR agonist is stable and can be efficiently released upon enzymatic activation.

### Example 2

### Design and Synthesis of Anti-CD11a IgGX-LXR Agonist ADC

Synthesis of the corresponding ADC was performed with the linker-derivatized LXR agonist from Example 1. To selectively deliver a LXR agonist to macrophages, CD11a was used as a target antigen. CD11a is the α-chain component of the lymphocyte function-associated antigen 1 (LFA-1). Although CD11a is expressed on most leukocytes, including lymphocytes and granulocytes, expression is abundant on monocytes and macrophages, and importantly, CD11a is not expressed on hepatocytes. Moreover, an increase in the expression of CD11a on monocytes is correlated with atherosclerotic coronary stenosis. CD11a receptors also internalize rapidly, and there are high affinity antibodies readily available (Kd˜2.2 nM) making it an attractive choice for this ADC.

In this study, unnatural amino acid (UAA) technology was utilized to incorporate a bio-orthogonal moiety (para-acetylphenylalanine, pAcF) site-specifically into anti-CD11a IgG for reaction with the terminal aminooxy group on our LXR agonist. Accordingly, the variable light and heavy regions of a humanized monoclonal anti-CD11a antibody were cloned into an expression vector containing the orthogonal aaRS/tRNA pair necessary for UAA incorporation in mammalian cells. pAcF was incorporated at position A122 in the constant region of the heavy chain.

Table 1 shows the DNA and protein sequences of the anti-CD11a A122pAcF IgG used in this experiment. The heavy chain DNA sequence for anti-CD11a A122pAcF IgG is SEQ ID NO.1 and the light chain DNA sequence for anti-CD11a IgG is SEQ ID NO. 2. The anti-CD11a A122pAcF IgG has a TAG where the UAA was incorporated (bold and underlined). anti-CD11a A122pAcF IgG heavy chain has a variable domain DNA sequence of SEQ ID NO. 3. anti-CD11a A122pAcF IgG has a CH1 domain DNA sequence of SEQ ID NO. 4. anti-CD11a A122pAcF IgG has a hinge DNA sequence of SEQ ID NO. 5. anti-CD11a A122pAcF IgG has a CH2 DNA sequence of SEQ ID NO. 6. anti-CD11a A122pAcF IgG has a CH3 DNA sequence of SEQ ID NO. 7. anti-CD11a A122pAcF IgG light chain has a variable domain DNA sequence of SEQ ID NO. 8. anti-CD11a A122pAcF IgG has a human light chain kappa DNA sequence of SEQ ID NO. 9.

Chinese Hamster Ovary (CHO) suspension cells were then transiently transfected with the expression vector, and the cells were grown for 7-10 days in pAcF-supplemented media to afford anti-CD11a A122pAcF IgG (referred to herein as anti-CD11a IgGX) in >4 mg/L yield (purified by Protein A chromatography). The heavy chain protein sequence for anti-CD11a A122pAcF IgG is SEQ ID NO.10 and the light chain protein sequence for anti-CD11a IgG is SEQ ID NO. 11. The anti-CD11a A122pAcF IgG has an “X” in Table 1 showing where the UAA was incorporated. anti-CD11a A122pAcF IgG heavy chain has a variable domain protein sequence of SEQ ID NO. 12. anti-CD11a A122pAcF IgG has a CH1 domain protein sequence of SEQ ID NO. 13. anti-CD11a A122pAcF IgG has a hinge protein sequence of SEQ ID NO. 14. anti-CD11a A122pAcF IgG has a CH2 protein sequence of SEQ ID NO. 15. anti-CD11a A122pAcF IgG has a CH3 protein sequence of SEQ ID NO. 16. anti-CD11a A122pAcF IgG light chain has a variable domain protein sequence of SEQ ID NO. 17. anti-CD11a A122pAcF IgG has a human light chain kappa protein sequence of SEQ ID NO. 18. The CDR1 of the anti-CD11a A122pAcF IgG heavy chain has the protein sequence of SEQ ID NO. 19. The CDR2 of the anti-CD11a A122pAcF IgG heavy chain has the protein sequence of SEQ ID NO. 20. The CDR3 of the anti-CD11a A122pAcF IgG heavy chain has the protein sequence of SEQ ID NO. 21. The CDR1 of the anti-CD11a A122pAcF IgG light chain has the protein sequence of SEQ ID NO. 22. The CDR2 of the anti-CD11a A122pAcF IgG light chain has the protein sequence of SEQ ID NO. 23. The CDR3 of the anti-CD11a A122pAcF IgG light chain has the protein sequence of SEQ ID NO. 24.

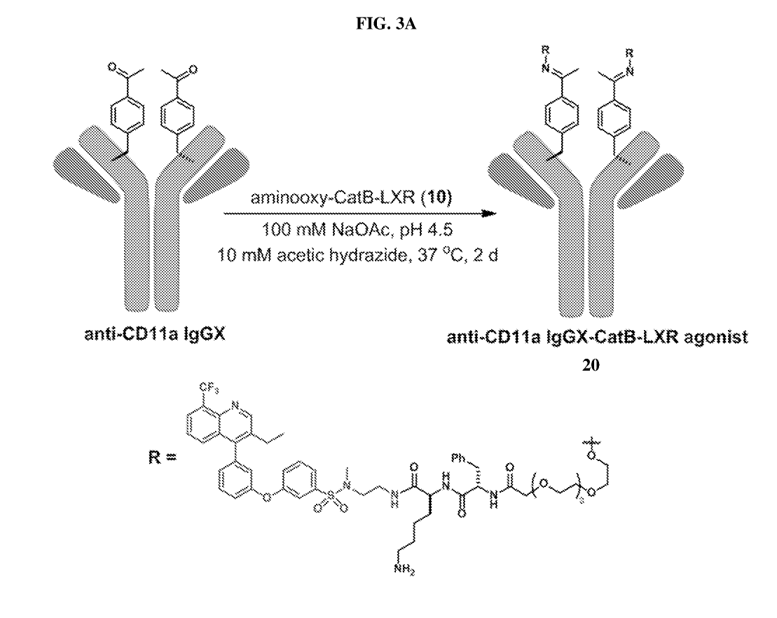

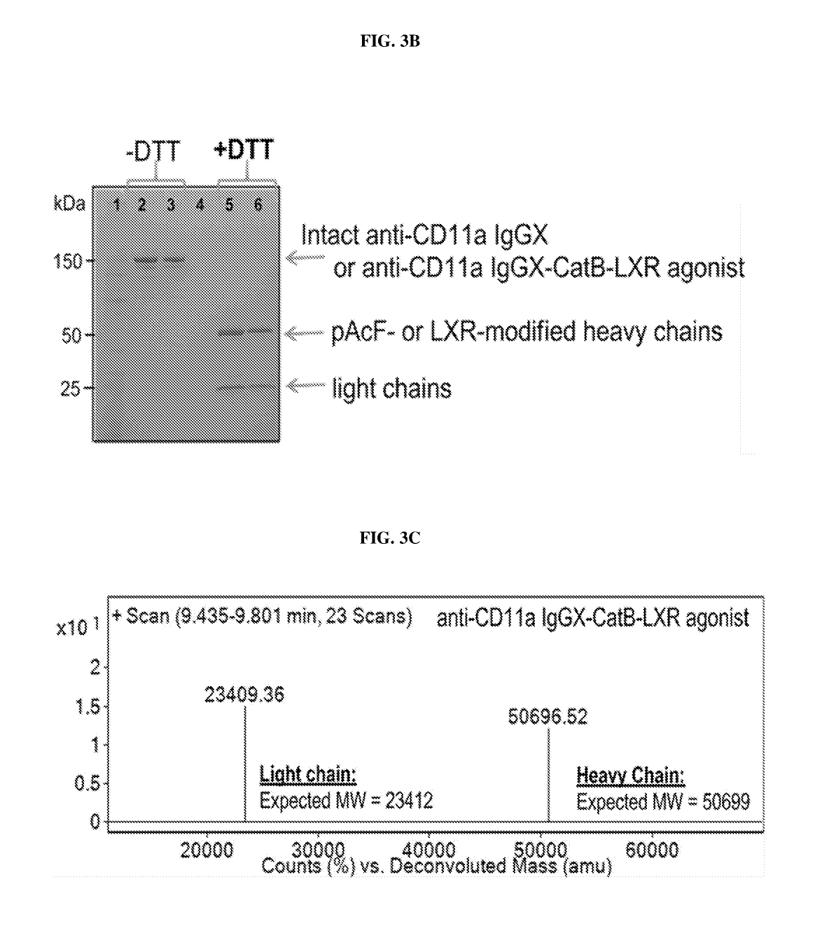

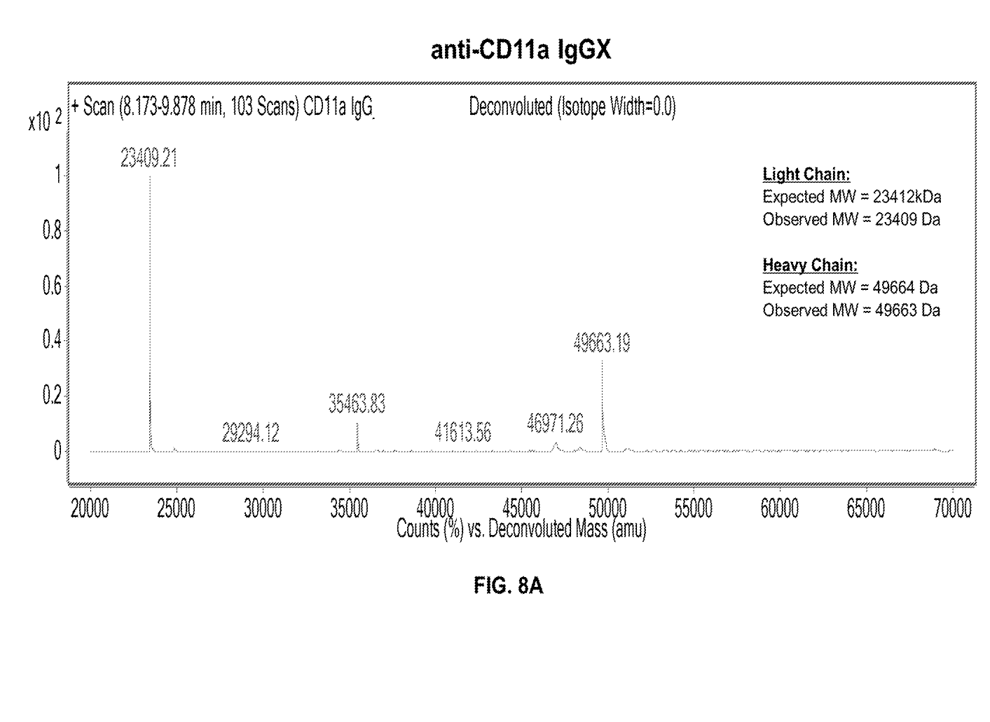

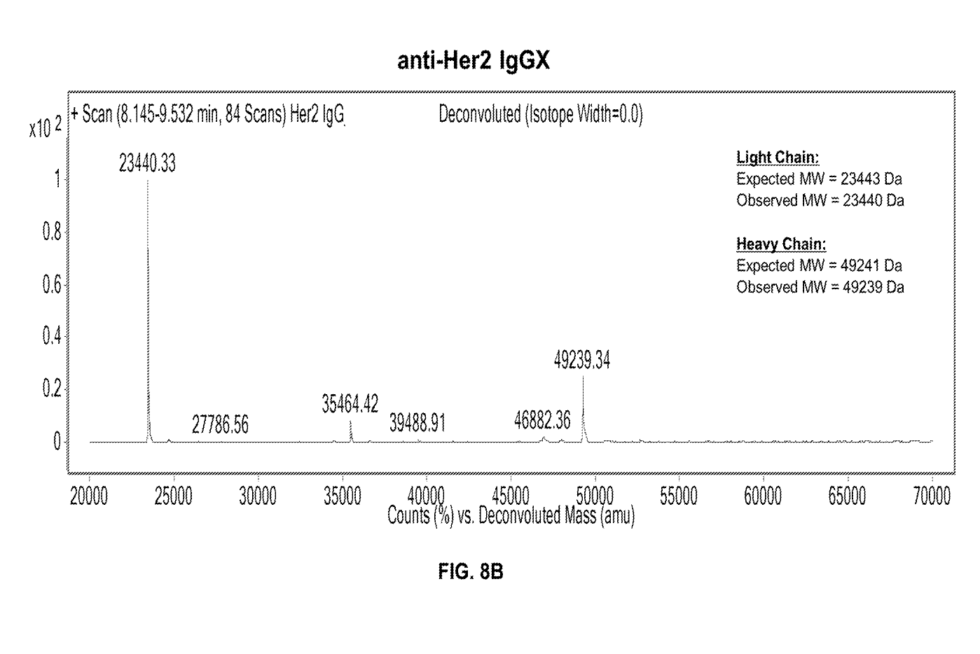

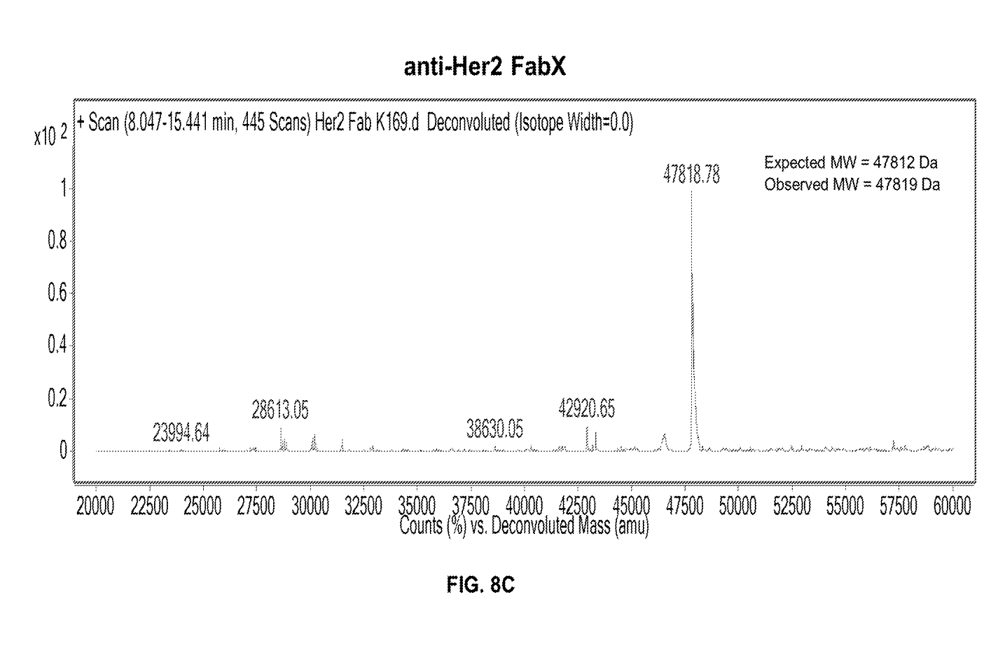

The identity and purity of the antibody was confirmed by denaturing SDS-PAGE gel electrophoresis (FIG. 3B) and electrospray-ionization mass spectrometry (ESI-MS) (FIG. 8A). Additionally, anti-Her2 A121pAcF IgG (anti-Her2 IgGX, FIG. 8B) and anti-Her2 K169pAcF Fab (anti-Her2 FabX, FIG. 8C) were expressed in CHO cells and E. coli, respectively, and were used as negative controls in subsequent assays.

Next, to covalently link the aminooxy-CatB-LXR agonist (compound 10) to anti-CD11a IgGX, anti-CD11a IgGX antibody (67-80 μM) was treated with 40 equivalents of compound 10 in sodium acetate buffer (pH 4.5) in the presence of 10 mM acetic hydrazide (catalyst) at 37° C. for 48 hours to generate anti-CD11a IgGX-CatB-LXR agonist (molecule 20, FIG. 3A). Excess compound 10 was removed by buffer exchange with an Amicon concentrator (30 kDa MWCO). Denaturing SDS-PAGE analysis of anti-CD11a IgGX-CatB-LXR agonist indicated that upon treatment with dithiothreitol (DTT), the conjugate gave rise to two bands with molecular weights corresponding to the heavy (˜50 kDa) and light chains (˜25 kDa) (FIG. 3B). As assessed by ESI-MS (FIG. 3C), the heavy chain peak had an increase in MW of ˜1030 Da (compared to unconjugated anti-CD11a IgGX, FIG. 8A) corresponding to the LXR agonist-modified product; and more than 95% of anti-CD11a IgGX was successfully coupled to the LXR agonist (FIG. 3C). Taken together, these results demonstrate the ability to site-specifically modify anti-CD11a IgGX with an LXR agonist, resulting in a chemically-defined, homogeneous product with a defined drug to antibody ratio (DAR) of 2.

TABLE 1
Antibody Sequences. Bold and underlined sequence indicates
location of unnatural amino acid in DNA sequences. ″X″ denotes
location of unnatural amino acid in protein sequences.

| | SEQ ID | |
|---|---|---|
| Name | NO. | Sequence |
| anti-CD11a | 1 | GAGGTGCAGCTGGTGGAATCTGGGGGGGGACTGGTGCAGCC |
| (A122pAcF) | | TGGCGGGTCACTGAGACTGTCCTGTGCCGCTTCTGGCTACTC |
| IgG Heavy | | TTTTACCGGCCACTGGATGAACTGGGTGCGACAGGCACCAG |
| Chain | | GCAAGGGACTGGAGTGGGTCGGAATGATCCATCCTTCTGAC |
| | | AGTGAAACACGGTACAATCAGAAGTTCAAAGACCGGTTCAC |
| | | CATTTCAGTGGATAAGAGCAAAAACACTCTGTACCTCCAGAT |
| | | GAACAGCCTGAGGGCCGAGGACACCGCTGTCTACTATTGCG |
| | | CCAGAGGCATCTACTTCTATGGAACCACATACTTTGATTATT |
| | | GGGGACAGGGCACTCTGGTGACCGTCAGCTCCTAGAGCACC |
| | | AAGGGCCCATCGGTCTTCCCCCTGGCACCCTCCTCCAAGAGC |
| | | ACCTCTGGGGGCACAGCGGCCCTGGGCTGCCTGGTCAAGGA |
| | | CTACTTCCCCGAACCGGTGACGGTGTCGTGGAACTCAGGCGC |
| | | CCTGACCAGCGGCGTGCACACCTTCCCGGCTGTCCTACAGTC |
| | | CTCAGGACTCTACTCCCTCAGCAGCGTGGTGACCGTGCCCTC |
| | | CAGCAGCTTGGGCACCCAGACCTACATCTGCAACGTGAATCA |
| | | CAAGCCCAGCAACACCAAGGTGGACAAGAAAGTTGAGCCCA |
| | | AATCTTGTGACAAAACTCACACATGCCCACCGTGCCCAGCAC |
| | | CTGAACTCCTGGGGGGACCGTCAGTCTTCCTCTTCCCCCCAA |
| | | AACCCAAGGACACCCTCATGATCTCCCGGACCCCTGAGGTCA |
| | | CATGCGTGGTGGTGGACGTGAGCCACGAAGACCCTGAGGTC |
| | | AAGTTCAACTGGTACGTGGACGGCGTGGAGGTGCATAATGC |
| | | CAAGACAAAGCCGCGGGAGGAGCAGTACAACAGCACGTACC |
| | | GTGTGGTCAGCGTCCTCACCGTCCTGCACCAGGACTGGCTGA |
| | | ATGGCAAGGAGTACAAGTGCAAGGTCTCCAACAAAGCCCTC |
| | | CCAGCCCCCATCGAGAAAACCATCTCCAAAGCCAAAGGGCA |
| | | GCCCCGAGAACCACAGGTGTACACCCTGCCCCCATCCCGGG |
| | | ATGAGCTGACCAAGAACCAGGTCAGCCTGACCTGCCTGGTC |
| | | AAAGGCTTCTATCCCAGCGACATCGCCGTGGAGTGGGAGAG |
| | | CAATGGGCAGCCGGAGAACAACTACAAGACCACGCCTCCCG |
| | | TGCTGGACTCCGACGGCTCCTTCTTCCTCTACAGCAAGCTCA |
| | | CCGTGGACAAGAGCAGGTGGCAGCAGGGGAACGTCTTCTCA |
| | | TGCTCCGTGATGCATGAGGCTCTGCACAACCACTACACGCAG |
| | | AAGAGCCTCTCCCTGTCTCCGGGTTGA |
| anti-CD11a | 2 | GACATTCAGATGACTCAGAGCCCTTCATCTCTGAGTGCCTCA |
| IgG Light | | GTGGGGGACCGGGTCACCATCACATGCAGAGCCAGCAAAAC |
| Chain | | AATTTCCAAGTACCTGGCTTGGTATCAGCAGAAGCCCGGCAA |
| | | AGCACCTAAGCTGCTGATCTACAGCGGCAGCACCCTCCAGTC |
| | | TGGAGTGCCCTCCAGGTTCTCTGGCAGTGGGTCAGGAACAGA |
| | | CTTTACTCTGACCATCAGCAGCCTCCAGCCAGAGGATTTCGC |
| | | TACTTACTATTGTCAGCAGCACAACGAATATCCCCTGACATT |
| | | TGGCCAGGGGACTAAAGTCGAGATCAAGCGTACGGTGGCTG |
| | | CACCATCTGTCTTCATCTTCCCGCCATCTGATGAGCAGTTGA |
| | | AATCTGGAACTGCCTCTGTTGTGTGCCTGCTGAATAACTTCT |
| | | ATCCCAGAGAGGCCAAAGTACAGTGGAAGGTGGATAACGCC |
| | | CTCCAATCGGGTAACTCCCAGGAGAGTGTCACAGAGCAGGA |
| | | CAGCAAGGACAGCACCTACAGCCTCAGCAGCACCCTGACGC |
| | | TGAGCAAAGCAGACTACGAGAAACACAAAGTCTACGCCTGC |
| | | GAAGTCACCCATCAGGGCCTGAGCTCGCCCGTCACAAAGAG |
| | | CTTCAACAGGGGAGAGTGT |
| anti-CD11a | 3 | GAGGTGCAGCTGGTGGAATCTGGGGGGGGACTGGTGCAGCC |
| (A122pAcF) | | TGGCGGGTCACTGAGACTGTCCTGTGCCGCTTCTGGCTACTC |
| IgG | | TTTTACCGGCCACTGGATGAACTGGGTGCGACAGGCACCAG |
| Variable | | GCAAGGGACTGGAGTGGGTCGGAATGATCCATCCTTCTGAC |
| Heavy | | AGTGAAACACGGTACAATCAGAAGTTCAAAGACCGGTTCAC |
| Chain | | CATTTCAGTGGATAAGAGCAAAAACACTCTGTACCTCCAGAT |
| | | GAACAGCCTGAGGGCCGAGGACACCGCTGTCTACTATTGCG |
| | | CCAGAGGCATCTACTTCTATGGAACCACATACTTTGATTATT |
| | | GGGGACAGGGCACTCTGGTGACCGTCAGCTCC |
| anti-CD11a | 4 | TAGAGCACCAAGGGCCCATCGGTCTTCCCCCTGGCACCCTCC |
| (A122pAcF) | | TCCAAGAGCACCTCTGGGGGCACAGCGGCCCTGGGCTGCCT |
| IgG CH1 | | GGTCAAGGACTACTTCCCCGAACCGGTGACGGTGTCGTGGA |
| | | ACTCAGGCGCCCTGACCAGCGGCGTGCACACCTTCCCGGCTG |
| | | TCCTACAGTCCTCAGGACTCTACTCCCTCAGCAGCGTGGTGA |
| | | CCGTGCCCTCCAGCAGCTTGGGCACCCAGACCTACATCTGCA |
| | | ACGTGAATCACAAGCCCAGCAACACCAAGGTGGACAAGAAA |
| anti-CD11a | 5 | GTTGAGCCCAAATCTTGTGACAAAACTCACACATGCCCACCG |
| (A122pAcF) | | TGCCCA |
| IgG Hinge | | |
| anti-CD11a | 6 | GCACCTGAACTCCTGGGGGGACCGTCAGTCTTCCTCTTCCCC |
| (A122pAcF) | | CCAAAACCCAAGGACACCCTCATGATCTCCCGGACCCCTGAG |
| IgG CH2 | | GTCACATGCGTGGTGGTGGACGTGAGCCACGAAGACCCTGA |
| | | GGTCAAGTTCAACTGGTACGTGGACGGCGTGGAGGTGCATA |
| | | ATGCCAAGACAAAGCCGCGGGAGGAGCAGTACAACAGCACG |
| | | TACCGTGTGGTCAGCGTCCTCACCGTCCTGCACCAGGACTGG |
| | | CTGAATGGCAAGGAGTACAAGTGCAAGGTCTCCAACAAAGC |
| | | CCTCCCAGCCCCCATCGAGAAAACCATCTCCAAAGCCAAA |
| anti-CD11a | 7 | GGGCAGCCCCGAGAACCACAGGTGTACACCCTGCCCCCATC |
| (A122pAcF) | | CCGGGATGAGCTGACCAAGAACCAGGTCAGCCTGACCTGCC |
| IgG CH3 | | TGGTCAAAGGCTTCTATCCCAGCGACATCGCCGTGGAGTGGG |
| | | AGAGCAATGGGCAGCCGGAGAACAACTACAAGACCACGCCT |
| | | CCCGTGCTGGACTCCGACGGCTCCTTCTTCCTCTACAGCAAG |
| | | CTCACCGTGGACAAGAGCAGGTGGCAGCAGGGGAACGTCTT |
| | | CTCATGCTCCGTGATGCATGAGGCTCTGCACAACCACTACAC |
| | | GCAGAAGAGCCTCTCCCTGTCTCCGGGTTGA |
| anti-CD11a | 8 | GACATTCAGATGACTCAGAGCCCTTCATCTCTGAGTGCCTCA |
| IgG | | GTGGGGGACCGGGTCACCATCACATGCAGAGCCAGCAAAAC |
| Variable | | AATTTCCAAGTACCTGGCTTGGTATCAGCAGAAGCCCGGCAA |
| Light Chain | | AGCACCTAAGCTGCTGATCTACAGCGGCAGCACCCTCCAGTC |
| | | TGGAGTGCCCTCCAGGTTCTCTGGCAGTGGGTCAGGAACAGA |
| | | CTTTACTCTGACCATCAGCAGCCTCCAGCCAGAGGATTTCGC |
| | | TACTTACTATTGTCAGCAGCACAACGAATATCCCCTGACATT |
| | | TGGCCAGGGGACTAAAGTCGAGATCAAG |
| anti-CD11a | 9 | CGTACGGTGGCTGCACCATCTGTCTTCATCTTCCCGCCATCTG |
| IgG Human | | ATGAGCAGTTGAAATCTGGAACTGCCTCTGTTGTGTGCCTGC |
| Kappa | | TGAATAACTTCTATCCCAGAGAGGCCAAAGTACAGTGGAAG |
| | | GTGGATAACGCCCTCCAATCGGGTAACTCCCAGGAGAGTGTC |
| | | ACAGAGCAGGACAGCAAGGACAGCACCTACAGCCTCAGCAG |
| | | CACCCTGACGCTGAGCAAAGCAGACTACGAGAAACACAAAG |
| | | TCTACGCCTGCGAAGTCACCCATCAGGGCCTGAGCTCGCCCG |
| | | TCACAAAGAGCTTCAACAGGGGAGAGTGT |
| anti-CD11a | 10 | EVQLVESGGGLVQPGGSLRLSCAASGYSFTGHWMNWVRQAPG |
| (A122pAcF) | | KGLEWVGMIHPSDSETRYNQKFKDRFTISVDKSKNTLYLQMNS |
| IgG Heavy | | LRAEDTAVYYCARGIYFYGTTYFDYWGQGTLVTVSSXSTKGPS |
| Chain | | VFPLAPSSKSTSGGTAALGCLVKDYFPEPVTVSWNSGALTSGVH |
| | | TFPAVLQSSGLYSLSSVVTVPSSSLGTQTYICNVNHKPSNTKVD |
| | | KKVEPKSCDKTHTCPPCPAPELLGGPSVFLFPPKPKDTLMISRT |
| | | PEVTCVVVDVSHEDPEVKFNWYVDGVEVHNAKTKPREEQYNST |
| | | YRVVSVLTVLHQDWLNGKEYKCKVSNKALPAPIEKTISKAKGQ |
| | | PREPQVYTLPPSRDELTKNQVSLTCLVKGFYPSDIAVEWESNGQ |
| | | PENNYKTTPPVLDSDGSFFLYSKLTVDKSRWQQGNVFSCSVMH |
| | | EALHNHYTQKSLSLSPG |
| anti-CD11a | 11 | DIQMTQSPSSLSASVGDRVTITCRASKTISKYLAWYQQKPGKAP |
| IgG Light | | KLLIYSGSTLQSGVPSRFSGSGSGTDFTLTISSLQPEDFATYYCQQ |
| Chain | | HNEYPLTFGQGTKVEIKRTVAAPSVFIFPPSDEQLKSGTASVVCL |
| | | LNNFYPREAKVQWKVDNALQSGNSQESVTEQDSKDSTYSLSST |
| | | LTLSKADYEKHKVYACEVTHQGLSSPVTKSFNRGEC |
| anti-CD11a | 12 | EVQLVESGGGLVQPGGSLRLSCAASGYSFTGHWMNWVRQAPG |
| (A122pAcF) | | KGLEWVGMIHPSDSETRYNQKFKDRFTISVDKSKNTLYLQMNS |
| IgG | | LRAEDTAVYYCARGIYFYGTTYFDYWGQGTLVTVSS |
| variable | | |
| anti-CD11a | 13 | XSTKGPSVFPLAPSSKSTSGGTAALGCLVKDYFPEPVTVSWNSG |
| (Al 22pAcF) | | ALTSGVHTFPAVLQSSGLYSLSSVVTVPSSSLGTQTYICNVNHKP |
| IgG CH1 | | SNTKVDKK |
| anti-CD11a | 14 | VEPKSCDKTHTCPPCP |
| (A122pAcF) | | |
| IgG Hinge | | |
| anti-CD11a | 15 | APELLGGPSVFLFPPKPKDTLMISRTPEVTCVVVDVSHEDPEVKF |
| (A122pAcF) | | NWYVDGVEVHNAKTKPREEQYNSTYRVVSVLTVLHQDWLNG |
| IgG CH2 | | KEYKCKVSNKALPAPIEKTISKAK |
| anti-CD11a | 16 | GQPREPQVYTLPPSRDELTKNQVSLTCLVKGFYPSDIAVEWESN |
| (A122pAcF) | | GQPENNYKTTPPVLDSDGSFFLYSKLTVDKSRWQQG |
| IgG CH3 | | NVFSCSVMHEALHNHYTQKSLSLSPG |
| anti-CD11a | 17 | DIQMTQSPSSLSASVGDRVTITCRASKTISKYLAWYQQKPGKAP |
| IgG | | KLLIYSGSTLQSGVPSRFSGSGSGTDFTLTISSLQPEDFATYYCQQ |
| Variable | | HNEYPLTFGQGTKVEIK |
| Light Chain | | |
| anti-CD11a | 18 | RTVAAPSVFIFPPSDEQLKSGTASVVCLLNNFYPREAKVQWKV |
| IgG Human | | DNALQSGNSQESVTEQDSKDSTYSLSSTLTLSKADYEKHKVYA |
| Kappa | | CEVTHQGLSSPVTKSFNRGEC |
| anti-CD11a | 19 | GYSFTGHWMN |
| CDR1HC | | |
| anti-CD11a | 20 | MIHPSDSETRYNQKFKD |
| CDR2HC | | |
| anti-CD11a | 21 | GIYFYGTTYFDY |
| CDR3HC | | |
| anti-CD11a | 22 | RASKTISKYLA |
| CDR1LC | | |
| anti-CD11a | 23 | SGSTLQS |
| CDR2LC | | |
| anti-CD11a | 24 | QQHNEYPLT |
| CDR3LC | | |

### Example 3

### Binding and Internalization of Anti-CD11a IgGX

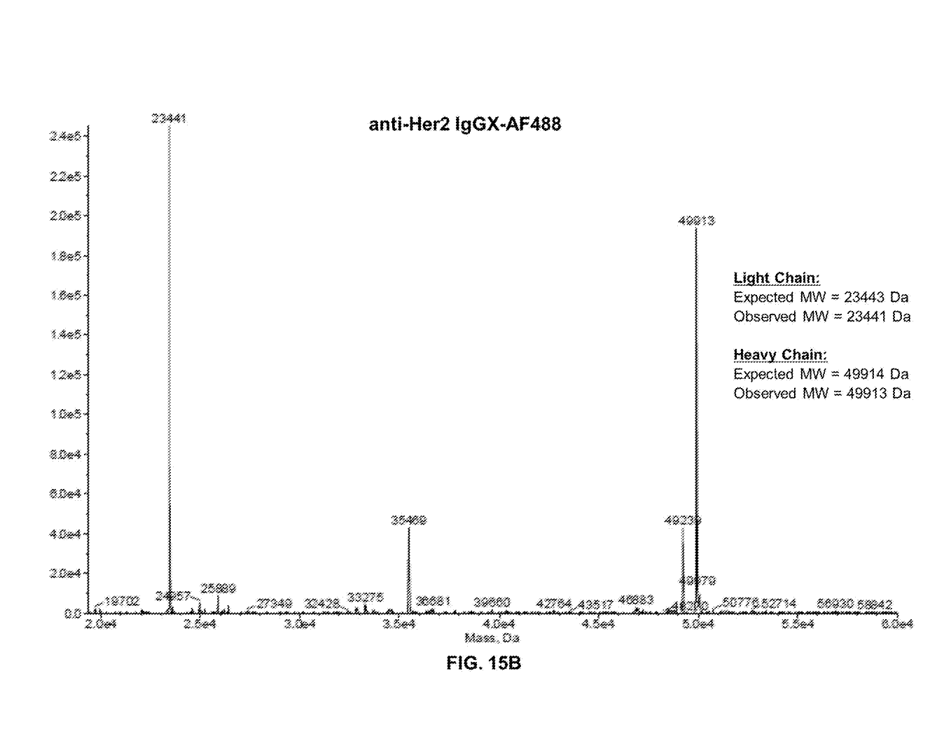

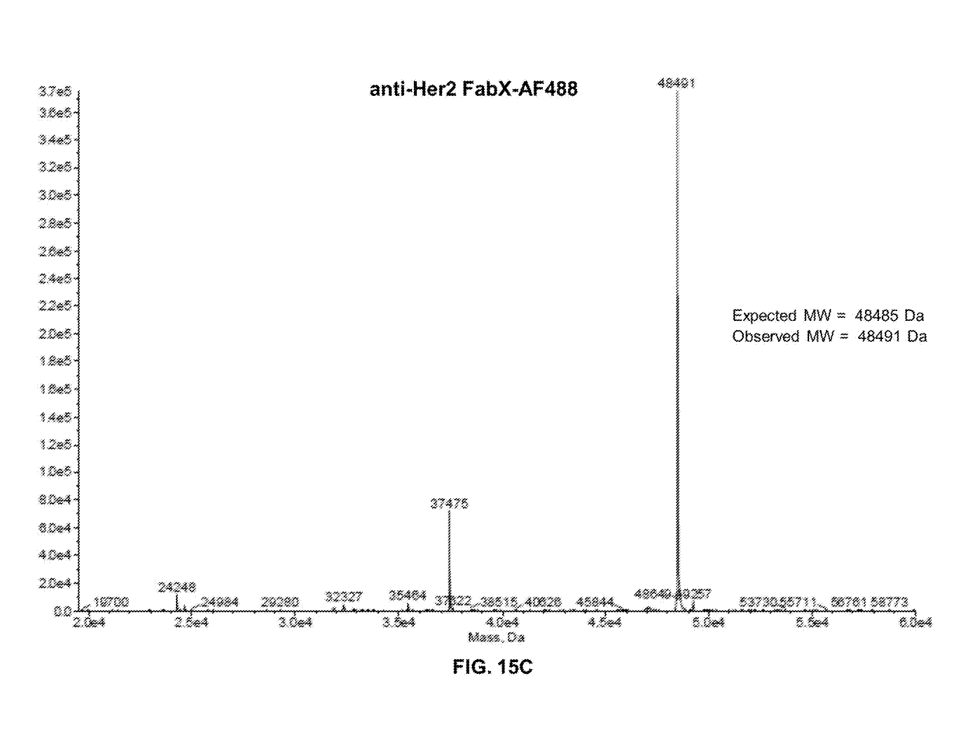

The affinity, specificity and internalization of the anti-CD11a IgGX-CatB-LXR agonist antibody (molecule 20, FIG. 3A) was determined. Anti-CD11a IgGX and negative controls (anti-Her2 IgGX and anti-Her2 FabX) were site-specifically conjugated to aminooxy-Alexa Fluor 488 (AF488, Life Technologies) as described in Example 2. FIG. 15A shows an ESI-MS spectra of anti-CD11a IgGX-AF488. FIG. 15B shows an ESI-MS spectra of anti-Her2 IgGX-AF488. FIG. 15C shows an ESI-MS spectra of anti-Her2 FabX-AF488.

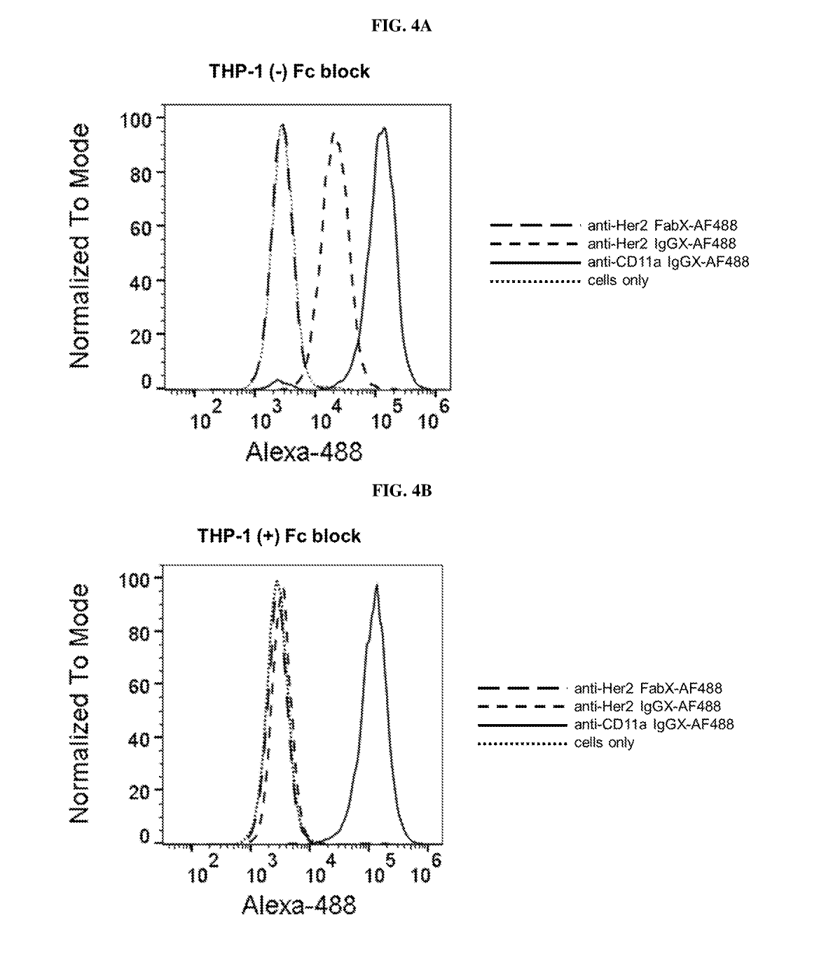

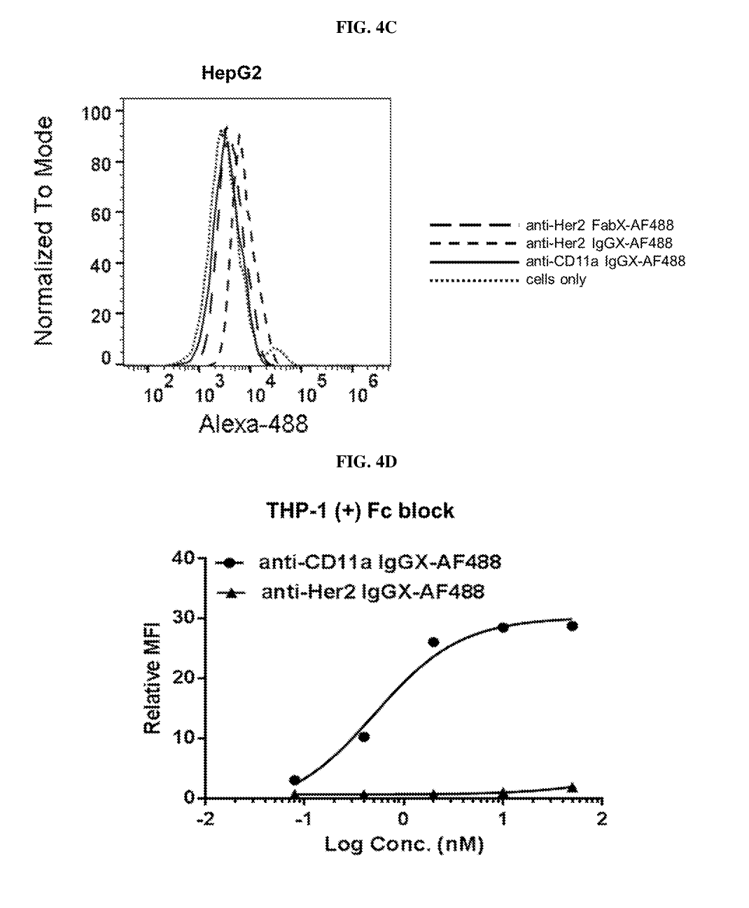

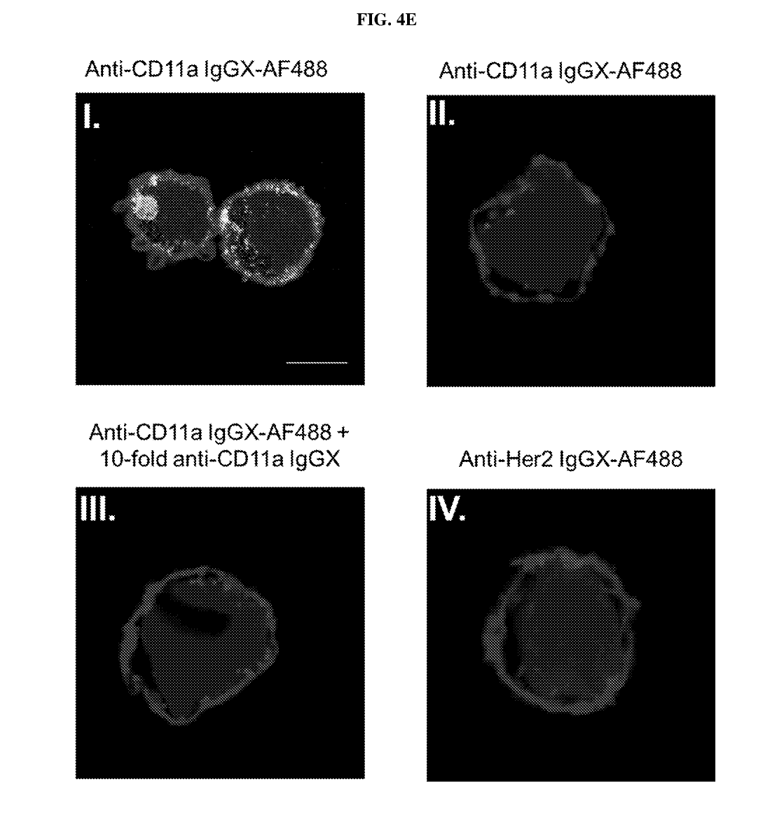

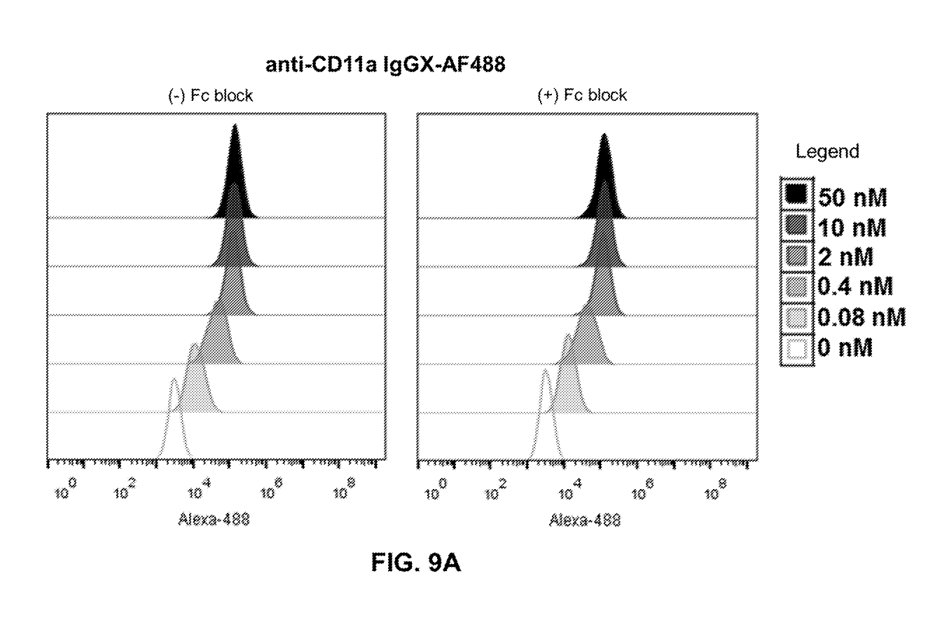

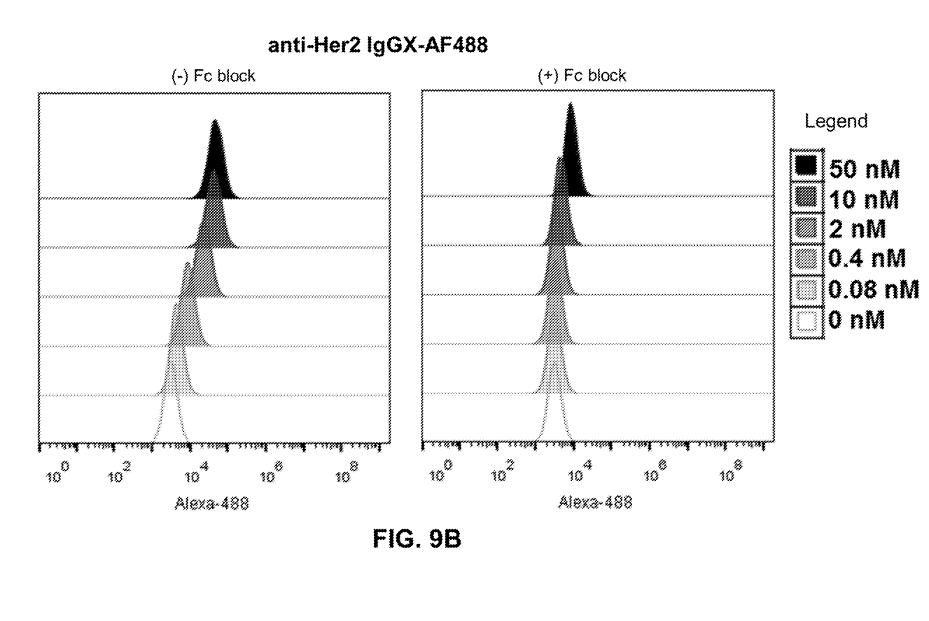

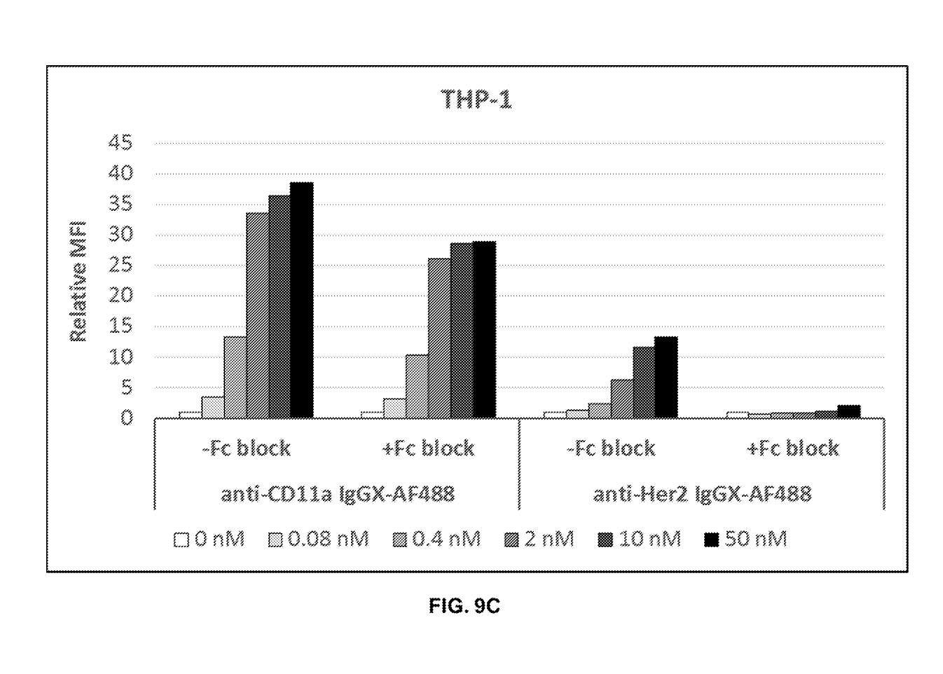

To assess the binding affinity of anti-CD11a IgGX and confirm it does not bind hepatocytes, human THP-1 monocyte/macrophage cells and human HepG2 hepatoma cells were incubated with anti-CD11a IgGX-AF488, anti-Her2 IgGX-AF488 and anti-Her2 FabX-AF488 and analyzed the results by flow cytometry. Because monocytes/macrophages have Fcγ receptors, the binding in the presence of an Fc blocking reagent was also examined (BD biosciences). As depicted in FIG. 4A, anti-CD11a IgGX-AF488 binds in the absence of Fc block, but still retains excellent binding affinity (Kd˜0.5 nM) once the Fc receptors are blocked (FIG. 4B), indicating the binding is CD11a-mediated (FIG. 4A and FIG. 9A). Conversely, anti-Her2 IgGX-AF488 also bound to THP-1 cells in the absence of Fc block (FIG. 4A), but did not bind once the Fc receptors were blocked (FIG. 4B), indicating that the binding of anti-Her2 IgG is Fc-mediated. To further verify this result, a fragment of Her2 (anti-Her2 FabX-AF488), which does not contain the Fc region was also tested. As expected, the anti-Her2 FabX-AF488 did not bind to THP-1 cells (FIG. 4A, FIG. 4B). FIG. 9C shows mean relative fluorescence intensities of the binding of AF488-conjugated antibodies to THP-1 cells in the presence or absence of Fc block. Furthermore, binding of anti-Her2 to THP-1 cells at different concentrations, with or without Fc block treatment was performed (FIG. 9B).

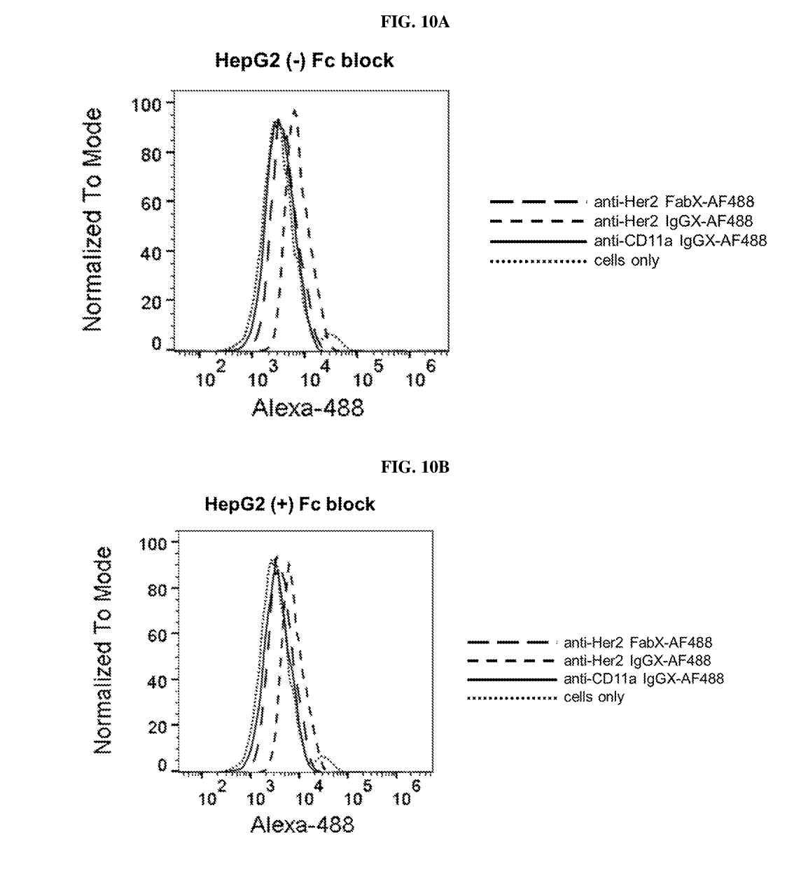

FIG. 4D shows binding specificity of anti-CD11a IgGX-AF488 and anti-Her2 IgGX-AF488 on THP-1(+) Fc block cells as shown by a plot of mean fluorescence intensity (MFI) versus concentration of antibody. Furthermore, incubation of anti-CD11a IgGX-AF488 with HepG2 cells did not result in any peak shift by flow cytometry (FIG. 4C), indicating anti-CD11a IgGX binds CD11a selectively and did not have an affinity towards hepatocytes (FIG. 10A, FIG. 10B).

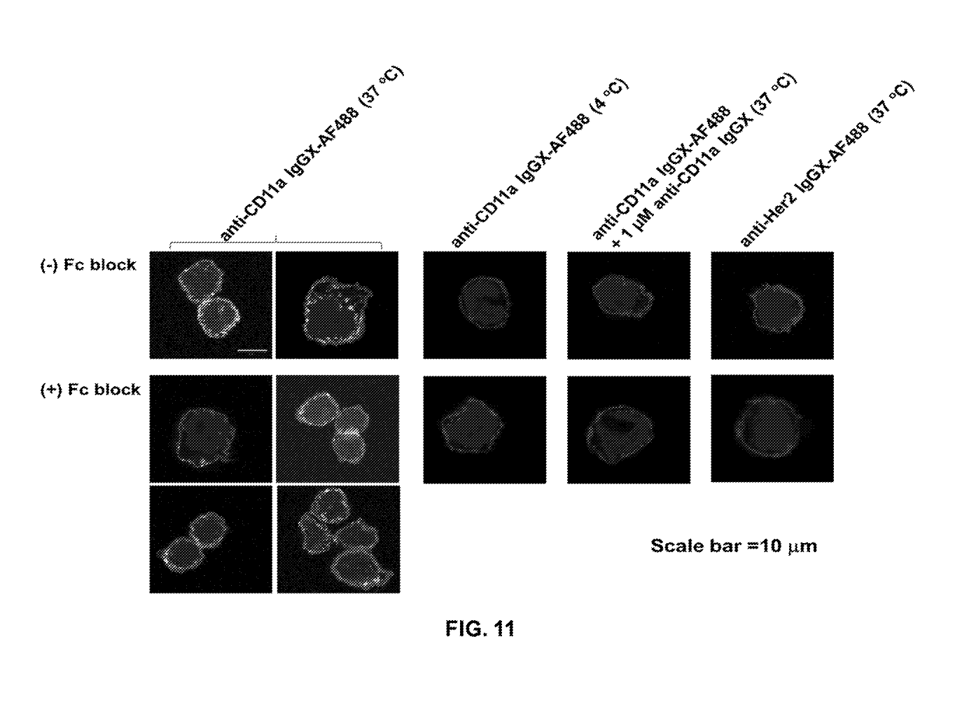

Given the high selectivity and affinity of anti-CD11a IgGX, its internalization by THP-1 cells, which is required for successful delivery of the LXR agonist, was examined. Confocal microscopy was used to determine the internalization efficiency of the anti-CD11a antibody into THP-1 cells in the presence or absence of Fc block (FIG. 4E and FIG. 11). As depicted in FIG. 4E, anti-CD11a IgGX-AF488 was observed in the cytoplasm within 2 hours at 37° C., which indicates efficient endocytosis. This effect was inhibited at 4° C. or in the presence of 20-fold excess of unconjugated anti-CD11a IgGX, which further confirms the internalization is CD11a-mediated. Anti-Her2 IgGX-AF488 was used as a negative control; no internalization was observed.

### Example 4

### In Vitro LXR Transactivation Assay

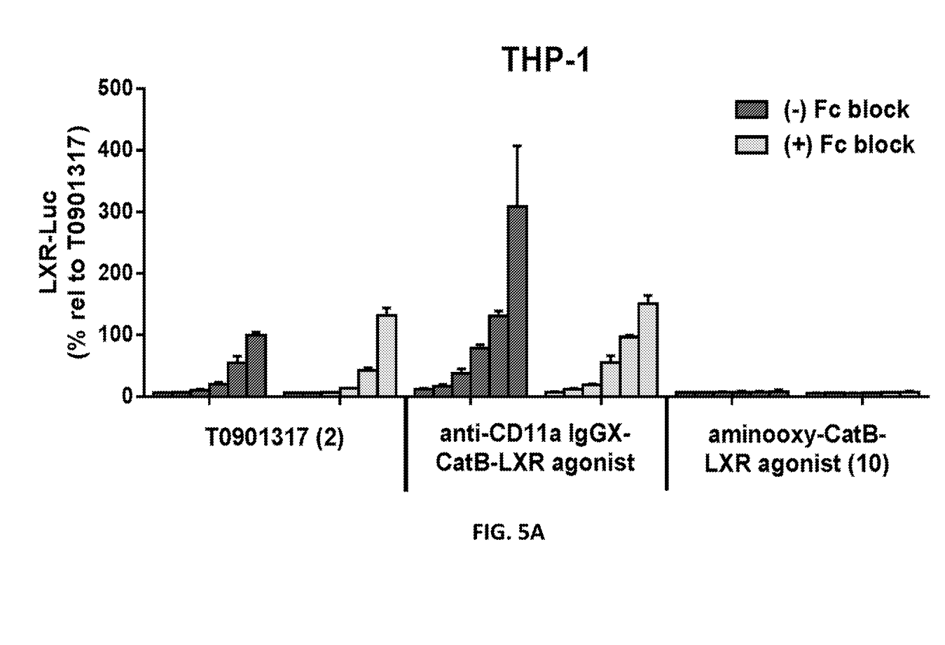

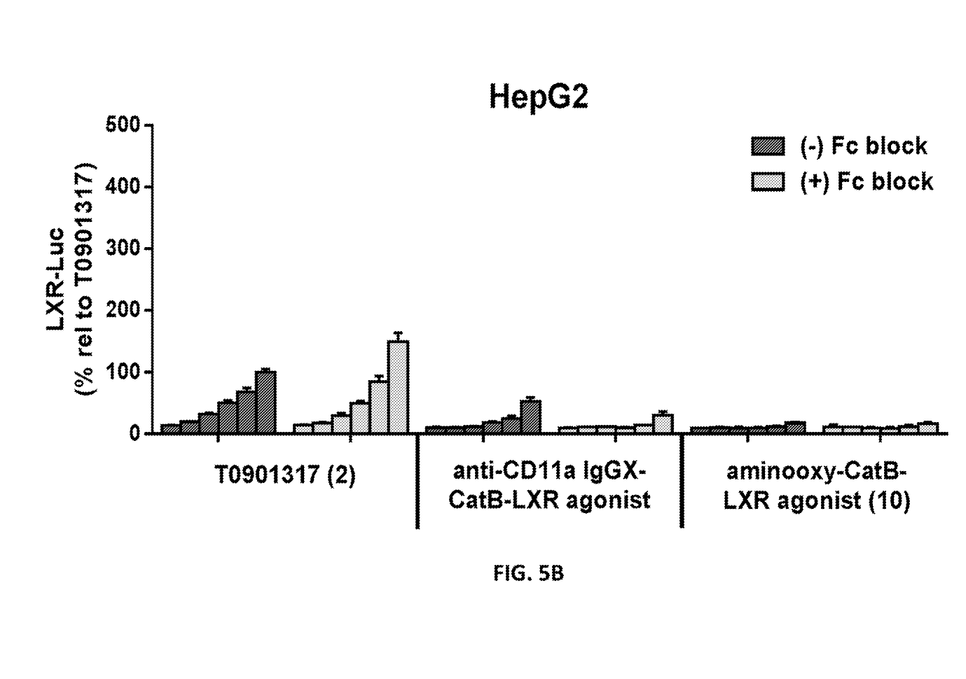

Next, the in vitro activity of the anti-CD11a IgGX-CatB-LXR agonist was evaluated using an LXR-driven luciferase stably incorporated into THP-1 and HepG2 cells. This vector encodes the firefly luciferase reporter gene under the control of a CMV promoter and tandem repeats of the LXR-α transcriptional response element. Cells were treated with anti-CD11a IgGX-CatB-LXR agonist, T0901317 (2, positive control) or aminooxy-CatB-LXR agonist (10) for 24 hours in the presence or absence of 10% human AB serum to block Fey receptors. Cells were then treated with ONE-Glo and luminescence was read on an Envision plate reader. Notably, LXR activation by the anti-CD11a IgGX-CatB-LXR agonist was selectively induced in THP-1 cells independent of Fc blockade (FIG. 5A). The ADC exhibited strong agonist activity with an EC50=26.7±1.3 nM compared to T0901317 (EC50=79.0±1.1 nM) in THP-1 cells. In HepG2 cells, anti-CD11a IgGX-CatB-LXR agonist did not induce significant activation (FIG. 5B), while T0901317 was equipotent in these cells relative to THP-1. Furthermore, no activity was detected in either cell type treated with compound 10, most likely due to a lack of cell permeability. These results indicate that the anti-CD11a ADC can selectively and effectively deliver a LXR agonist to THP-1 cells.

### Example 5

### Chemical Syntheses

See FIGS. 12-14 for chemical structures that correspond to the compound numbers (bold) in the following descriptions.

Dipeptide 13.

To a solution of H-Lys(Boc)-OMe.HCl 11 (500 mg, 1.68 mmol) and N-Cbz-L-Phenylalanine 12 (605 mg, 2.02 mmol) in DMF (6 mL) was added DIPEA (1.17 mL, 6.72 mmol), HOBt.H₂O (80%, 645 mg, 3.37 mmol) and EDCI (484 mg, 2.52 mmol). The reaction was stirred for 16 h at room temperature, and then partitioned between DCM and 1 N HCl. The organic phase was collected, washed with saturated NaHCO₃ and brine, and then dried over Na₂SO₄ and concentrated. The crude product was purified by silica gel chromatography (4:1 to 1:1, hexanes/EtOAc) to give 860 mg (91%) dipeptide 13. ¹H NMR (400 MHz, CDCl₃) δ 7.37-7.24 (m, 8H), 7.19 (app d, J=6.8 Hz, 2H), 6.34 (app d, J=7.2 Hz, 1H), 5.37 (br s, 1H), 5.09 (s, 2H), 4.64 (br s, 1H), 4.52 (dd, J=12.4, 7.6 Hz, 1H), 4.43 (dd, J=14.0, 7.2 Hz, 1H), 3.70 (s, 3H), 3.10-3.03 (m, 4H), 1.82-1.74 (m, 1H), 1.65-1.56 (m, 2H), 1.46-1.37 (m, 10H), 1.26-1.17 (m, 2H).

Amine 14.

Dipeptide 13 (270 mg, 0.50 mmol) was dissolved in degassed MeOH (5 mL) containing 10% Pd/C (53 mg), and the mixture was stirred under H₂ (1 atm) for 18 h at room temperature. The reaction was filtered through a pad of celite, and the pad was washed with MeOH. The combined filtrates and washings were concentrated under reduced pressure to give 0.18 g (90%) crude 14 that was used directly in the next step without further purification. ¹H NMR (400 MHz, CDCl₃) δ 7.78 (d, J=8.0 Hz, 1H), 7.34-7.22 (m, 5H), 4.61-4.56 (m, 2H), 3.73 (s, 3H), 3.66 (dd, J=9.2, 4.4 Hz, 1H), 3.25 (dd, J=12.0, 4.0 Hz, 1H), 3.09 (app dd, J=12.8, 6.4 Hz, 2H), 2.74 (dd, J=16.0, 8.0 Hz, 1H), 1.88-1.79 (m, 1H), 1.72-1.62 (m, 1H), 1.50-1.39 (m, 11H), 1.32-1.23 (m, 2H).

Ester 16.

To a suspension of NaH (60% in mineral oil, 0.22 g, 5.5 mmol) in THF (8 mL) was added a solution of tetraethylene glycol monobenzyl ether 15 (1.33 g, 4.68 mmol) in THF (4 mL) at 0° C. After being stirred for 25 min at 0° C., methyl bromoacetate (0.58 mL, 6.12 mmol) was added dropwise to the reaction. The reaction was stirred for 1 h at 0° C., and then warmed to room temperature and stirred overnight. The reaction was carefully quenched with MeOH, and filtered through a pad of celite, and the pad was washed with MeOH. The combined filtrates and washings were concentrated under reduced pressure. The crude product was purified by silica gel chromatography (4:1 to 2:1, hexanes/EtOAc) to give 1.11 g (66%) 16. ¹H NMR (400 MHz, CDCl₃) δ 7.34 (d, J=4.4 Hz, 4H), 7.31-7.27 (m, 1H), 4.56 (s, 2H), 4.16 (s, 2H), 3.76-3.61 (m, 19H).

Acid 17.

Ester 16 (0.28 g, 0.79 mmol) and LiOH (38 mg, 1.58 mmol) was stirred in a mixture of THF (2 mL)/H₂O (2 mL) at room temperature for 2 h. Then the reaction was quenched with 1 N HCl (2 mL), and partitioned between EtOAc and brine. The organic phase was collected, the aqueous phase was extracted with EtOAc. The combined organic layers were dried over Na₂SO₄, filtered and concentrated to give 0.26 g (96%) crude 17 that was used directly in the next step without further purification. ¹H NMR (400 MHz, CDCl₃) δ 7.34 (d, J=4.4 Hz, 4H), 7.31-7.26 (m, 1H), 4.57 (s, 2H), 4.13 (s, 2H), 3.75-3.72 (m, 2H), 3.70-3.62 (m, 14H).

Benzyl Ether 18.

To a solution 17 (0.18 g, 0.052 mmol) and 14 (0.18 g, 0.044 mmol) in DMF (6 mL) was added DIPEA (0.3 mL, 1.72 mmol), HOBt.H₂O (80%, 0.25 g, 1.30 mmol) and EDCI (0.17 g, 0.89 mmol). The reaction was stirred for 16 h at room temperature, and then partitioned between EtOAc and 1 N HCl. The organic phase was collected, washed with saturated NaHCO₃ and brine, and then dried over Na₂SO₄ and concentrated. The crude product was purified by silica gel chromatography (50:1, DCM/MeOH) to give 290 mg (90%) compound 18. ¹H NMR (400 MHz, CDCl₃) δ 7.38-7.21 (m, 10H), 6.52 (d, J=7.6 Hz, 1H), 4.75 (br s, 1H), 4.64 (dd, J=14.8, 7.6 Hz, 1H), 4.55 (s, 2H), 4.48 (dd, J=13.2, 7.6 Hz, 1H), 3.96 (dd, J=30.4, 16.0 Hz, 2H), 3.69-3.53 (m, 18H), 3.18-3.04 (m, 4H), 1.84-1.58 (m, 2H), 1.47-1.41 (m, 11H), 1.31-1.21 (m, 2H).

Alcohol 19.

Compound 18 (280 mg, 0.38 mmol) was dissolved in degassed MeOH (6 mL) containing 10% Pd/C (40 mg), and the mixture was stirred under H₂ (1 atm) for 18 h at room temperature. The reaction was filtered through a pad of celite, and the pad was washed with MeOH. The combined filtrates and washings were concentrated under reduced pressure to give 188 mg (76%) crude 19 that was used directly in the next step without further purification. ¹H NMR (400 MHz, CDCl₃) δ 7.59 (app d, J=6.4 Hz, 1H), 7.30-7.19 (m, 5H), 6.69 (app d, J=10.0 Hz, 1H), 4.83 (br s, 1H), 4.65 (dd, J=14.8, 8.0 Hz, 1H), 4.49 (app q, J=13.2, 7.6 Hz, 1H), 3.97 (dd, J=36.0, 15.6 Hz, 2H), 3.72-3.51 (m, 18H), 3.20-3.05 (m, 4H), 1.83-1.75 (m, 1H), 1.68-1.58 (m, 1H), 1.45 (app s, 11H), 1.31-1.24 (m, 2H).

Tosylate 20.

To a solution of 19 (428 mg, 0.67 mmol) in CH₃CN (10 mL) was added Et₃N (0.19 mL, 1.36 mmol) and TsCl (190 mg, 1.0 mmol). The reaction was stirred overnight at room temperature, and then concentrated. The crude product was purified by silica gel chromatography (50:1 to 10:1, DCM/MeOH) to give 310 mg (58%) compound 20. ¹H NMR (400 MHz, CDCl₃) δ 7.75 (d, J=8.4 Hz, 2H), 7.35 (d, J=8.0 Hz, 1H), 7.30 (d, J=8.0 Hz, 2H), 7.25-7.16 (m, 5H), 6.69 (d, J=7.6 Hz, 1H), 4.81 (br s, 1H), 4.65 (dd, J=14.8, 7.6 Hz, 1H), 4.44 (dd, J=13.2, 7.6 Hz, 1H), 4.10 (app t, J=4.8 Hz, 2H), 3.92 (dd, J=32.0, 15.6 Hz, 2H), 3.65 (s, 3H), 3.63-3.51 (m, 14H), 3.11 (dd, J=14.0, 6.4 Hz, 1H), 3.04-2.99 (m, 1H), 2.40 (s, 3H), 1.80-1.71 (m, 1H), 1.64-1.55 (m, 1H), 1.39 (app s, 10H), 1.29-1.18 (m, 3H).

Acid 9.

Tosylate 20 (310 mg, 0.39 mmol) and LiOH (38 mg, 0.79 mmol) was stirred in a mixture of THF (5 mL)/H₂O (2.5 mL) at room temperature for 2 h. Then the reaction was quenched with 1 N HCl (1 mL), and partitioned between EtOAc and brine. The organic phase was collected, the aqueous phase was extracted with EtOAc. The combined organic layers were dried over Na₂SO₄, filtered and concentrated to give 300 mg (98%) crude 9 that was used directly in the next step without further purification. LC-MS: t_{R} 3.83 min, m/z (ES, [M+H]⁺) 782.3.

Sulfonamide 8.

To a mixture of 6¹ (117 mg, 0.37 mmol) and 7² (163 mg, 0.55 mmol) in dioxane (2 ml) was added Me₂NCH₂COOH hydrochloride (24 mg, 0.17 mmol), Cs₂CO₃ (162 mg, 0.5 mmol) and CuI (11 mg, 0.058 mmol). The reaction was heated to 120° C. for 48 h in a sealed tube. After cooling to room temperature, the mixture was partitioned between EtOAc and water. The organic phase was collected, dried over Na₂SO₄ and concentrated. The crude product was purified by silica gel chromatography (2:1 to 1:1, Hexanes/EtOAc) to give 89 mg (45%) sulfonamide 8. LC-MS: t_{R} 4.61 min, m/z (ES, [M+H]⁺) 531.2.

Mesylate 21.

To a solution of sulfonamide 8 (89 mg, 0.17 mmol) in DCM (5 mL) was added DIPEA (0.2 mL, 1.15 mmol) and MsCl (66 μL, 0.85 mmol). The reaction was stirred for 2.5 h at room temperature, and then quenched with H₂O. The mixture was partitioned between DCM and water. The organic phase was collected, dried over Na₂SO₄ and concentrated. The crude product was purified by silica gel chromatography (4:1 to 1:1, Hexanes/EtOAc) to give 51 mg (50%) mesylate 21. LC-MS: t_{R} 4.70 min, m/z (ES, [M+H]⁺) 609.2.

Amine 3.

Mesylate 21 (98 mg, 0.16 mmol) was dissolved in 7 N NH₃ in MeOH (6 mL) in a sealed tube. The reaction was heated to 50° C. for 48 h. After cooling to room temperature, the reaction mixture was concentrated to give 78 mg (91%) crude amine 3 that was used directly in the next step without further purification. LC-MS: t_{R} 3.62 min, ink (ES, [M+H]⁺) 530.2. Amine 3 HCl salt. ¹H NMR (400 MHz, DMSO) δ 9.08 (s, 1H), 8.14 (t, J=3.6 Hz, 1H), 8.03 (br s, 3H), 7.72-7.65 (m, 4H), 7.53 (d, J=8.0 Hz, 1H), 7.47 (dd, J=8.0, 2.0 Hz, 1H), 7.38 (m, 1H), 7.31 (dd, J=8.0, 2.0 Hz, 1H), 7.23 (d, J=7.6 Hz, 1H), 7.152 (m, 1H), 3.16 (t, J=6.4 Hz, 2H), 2.99-2.92 (m, 2H), 2.69 (s, 3H), 2.61 (q, J=7.6 Hz, 2H), 1.11 (t, J=7.6 Hz, 3H).

Tosylate 22.

To a mixture of 3 (78 mg, 0.147 mmol) and 9 (116 mg, 0.147 mmol) in DMF (3 mL) was added DIPEA (0.1 mL, 0.57 mmol), HOBt.H₂O (80%, 85 mg, 0.44 mmol) and EDCI (57 mg, 0.27 mmol). The reaction was stirred for 20 h at room temperature, and then partitioned between DCM and 1 N HCl. The organic phase was collected, washed with saturated NaHCO₃ and brine, and then dried over Na₂SO₄ and concentrated. The crude product was purified by silica gel chromatography (20:1 to 10:1, DCM/MeOH) to give 97 mg (51%) tosylate 22. LC-MS: t_{R} 5.22 min, ink (ES, [M+H]⁺) 1293.6.

Compound 23.

To a mixture of 22 (97 mg, 0.075 mmol) and N-hydroxyphthalimide (15 mg, 0.092 mmol) in DMF (5 mL) was added DBU (16 μL, 0.11 mmol). After stirring for 20 h in a sealed tube at 50° C., the reaction was quenched with 1 N HCl. The mixture was partitioned between EtOAc and H₂O. The organic phase was collected, and the aqueous phase was extracted with EtOAc. The combined organic layers were dried over Na₂SO₄ and concentrated. The crude product was purified by silica gel chromatography (20:1 to 10:1, DCM/MeOH) to give 66 mg (69%) compound 23. LC-MS: t_{R} 4.92 min, ink (ES, [M+H]⁺) 1284.5.

Hydroxylamine 10.

To a solution of compound 23 (66 mg, 0.05 mmol) in DCM (3 mL) was added TFA (1.5 mL). The reaction was stirred for 3 h at room temperature, and then concentrated to give 80 mg crude product. The crude product was dissolved in DCM, and N₂H₄.H₂O (17 μL, 0.35 mmol) was added. After stirring overnight at room temperature, the solvent was removed by reduced pressure. The crude product was dissolved in CH₃CN and purified by reverse phase preparative HPLC (solvent B from 10% to 90% over a time period of 6 minutes at a flow rate of 60 mL/min) to give 15 mg (27%) hydroxylamine 10 over two steps. ¹H NMR (400 MHz, CD₃OD) δ 8.96 (s, 1H), 8.20-8.06 (m, 2H), 7.71-7.57 (m, 4H), 7.52 (app d, J=8.0 Hz, 1H), 7.40-7.37 (m, 2H), 7.29-7.17 (m, 6H), 7.03 (t, J=1.6 Hz, 1H), 4.61-4.56 (m, 1H), 4.38-4.32 (m, 1H), 4.09 (app t, J=4.8 Hz, 2H), 3.97 (dd, J=30.4, 15.6 Hz, 2H), 3.68-3.58 (m, 12H), 3.17 (dd, J=13.6, 6.4 Hz, 1H), 3.07-2.92 (m, 4H), 2.72-2.66 (m, 4H), 1.96-1.88 (m, 1H), 1.82 (d, J=2.0 Hz, 4H), 1.73-1.60 (m, 3H), 1.52-1.44 (m, 2H), 1.33-1.24 (m, 2H), 1.20-1.15 (m, 3H). LC-MS: t_{R} 3.45 min, m/z (ES, [M+H]⁺) 1054.4.

### Example 6

### Synthesis of Alexa Fluor 488-Antibody Conjugates

A solution of pAcF-containing antibody [0.5 mg, 3.3 nmol (IgG, SEQ ID NO. 10 and 11) or 10 nmol (Fab)] in 100 mM sodium acetate buffer, pH 4.5 (final concentration of antibody ranges from 5-10 mg/mL) was reacted with 25-40 equiv of the Alexa Fluor 488 hydroxylamine (Life Technologies). The mixture was left to react at 37° C. for 48 h in the dark. Afterwards, the dye-conjugated antibody was isolated from excess small molecule using Zeba spin desalting column [MWCO: 40 kDa (IgG), 7 kDa (Fab), Thermo Scientific] and/or Amicon filter [MWCO: 30 kDa (IgG), 10 kDa (Fab), EMD Millipore). The identities of all the resulting dye-conjugated antibodies were confirmed by ESI-MS.

### Example 7

### LanthaScreen Cell-Based Binding Assay

The kits used in this assay were: LanthaScreen TR-FRET LXR alpha Coactivator Assay Kit, goat (Cat. No PV4655, Life Technologies) and LanthaScreen TR-FRET LXR beta Coactivator Assay Kit, goat (Cat. No PV4658, Life Technologies). First, the LXR agonist compounds were transferred (20 nL of 10 mM, followed by 1:3 serial dilution) into blank 384 LDV NBS microplates (Corning 4514) by Echo liquid handler (Labcyte, Inc.). Next, 10 μL of the completed TR-FRET Co-regulator buffer (supplemented with 5 mM DTT) was added on top of the compounds. Afterwards, the plates were shaken to ensure proper mixing of the compounds. Also, control wells were included that contain 0.1% DMSO only. Both 4×LXR alpha (or beta)-ligand binding domain (LBD) and 4×fluorescein/Tb anti-GST antibody solution were prepared following the manufacturer's protocol. Next, 5 μL of the 4×LXR alpha (or beta)-LBD solution was added to each well and into the DMSO control. After shaking the plates, 5 μL of the 4×fluorescein/Tb anti-GST antibody solution was also added to each well including the DMSO control. At this point onwards, all plates were protected from light. After final shaking, the plates were sealed and incubated for at least 1 h at room temperature. All plates were read using the EnVision plate reader (Perkin Elmer) between 1-6 h based on the following settings: Excitation wavelength: 340 nM (30 nm bandwidth); Emission wavelengths: 490 or 495 nm (10 nm bandwidth) and 520 nm (25 nm bandwidth). The emission ratio of 520 nm to 490 nm was calculated and normalized in Genedata. A dose-response curve was then generated, and EC₅₀ was calculated based on the fitted curve.

### Example 8

### Compound Stability Testing

Compound 10 (Final concentration of 50 μg/mL, aminoxy group was capped (capped 10, FIG. 7B) with acetone to prevent occurrence of undesirable reaction) was added individually to 48 μL of THP-1 growth media (RPMI+10% PBS+sodium pyruvate+1% Pen/Strep+β-mercaptoethanol) and also into 48 μL of sodium heparin-treated female CD-1 mouse plasma (BioreclamationIVT, New York). Final volume for all samples consisted of 50 μL. Samples were incubated at 37° C. at various time points (0, 10 min, 0.5 h, 1 h, 2 h, 4 h, 8 h, and 24 h). Samples were extracted with 350 μL ice cold acetonitrile containing internal standard.

2.5 μL Cathepsin-B (Lot# D00170366, EMD Millipore, MA) was activated in 25 μL 50 mM sodium acetate buffer containing 1 mM EDTA and 2 mM DTT at 22° C. for 15 min prior to the addition of compound 10 (final concentration of 5 μM). Total volume of samples was 29 μL. Samples were incubated at 37° C. at various time points corresponding to the growth media- and plasma-incubated samples. Samples were extracted with 200 μL ice cold acetonitrile containing internal standard.

LC-MS analysis was done using Applied Biosystems 4000 Q-Trap mass spectrometer with an Agilent 1100 LC utilizing positive mode Electrospray ionization and the Analyst 1.6.2 software package. Detection of the molecular ions [M+H] and fragment ions was conducted by separation utilizing a Luna C-18(2) column (5u, 50 mm×2.0 mm) (Phenomenex, CA). Mobile phase used consisted of solvent A (water/0.01% formic acid) and solvent B (acetonitrile/0.01% formic acid) at a flow rate of 0.5 mL/min. The elution gradient changed linearly from 2 min to 3.5 min, from 90%/10% (A/B) to 5%/95% (A/B). The gradient was held for 1.5 min until it returns to 90% A. Total analysis run time is 7 min. Incubated samples were compared against the control samples, and percent changes were calculated by comparison of the loss of parent compound 10.

### Example 9

### Construction of Stable Cell Lines for LXR Transactivation Assay

THP-1 cells and HepG2 cells (ATCC) were seeded at 0.6×10⁶ cells/well in Opti-MEM media in a 6-well plate and allowed to incubate overnight at 37° C. Afterwards, 50 μL of Cignal lenti LXR-α luciferase reporter (cat no: CLS-7041L, Qiagen) was added to the wells with gentle mixing. After 48 h, the media was changed to completed growth media that was supplemented with 1 μg/mL puromycin. After 24 h, positively transfected cells were cultured for one week in puromycin-containing media. Finally, the antibiotics were removed from the media and the selected cells were recovered and cultured in the corresponding completed growth media for 2-3 days before the assay.

### Example 10

### Expression/Purification of pAcF Antibodies

Anti-CD11a A122pAcF IgG and anti-Her2 IgG A121pAcF IgG were expressed in suspension CHO cells (CHO-S, Life Technologies) by transient incorporation of tRNA/aaRS pair and antibody gene as previously described. After 7-10 days the supernatant was harvested and filtered (0.22 μm) before passing through a Protein A column previously equilibrated with 1×PBS. Antibodies were eluted with 100 mM glycine (pH 2.8) and later neutralized with 10% volume of 1 M Tris-HCl (pH 8.0). Antibodies were then buffer exchanged and concentrated into 1×PBS (Amicon, 30 kDA MWCO). Anti-Her2 K169pAcF Fab was expressed in E. coli as previously described and purified by Protein G chromatography.

### Example 11

### Confocal Microscopic Analysis

THP-1 cells (2×10⁶) were suspended in an eppendorf tube containing 600 μL RPMI media supplemented with 10% FBS and 1% penicillin/streptomycin. Before treatment with antibodies, cells were either incubated with or without human Fc block (0.0125 mg/mL, final concentration, BD Biosciences) for 20 min at room temperature. Next, cells were treated with 50 nM (final concentration) of anti-CD11a IgGX-AF488 or anti-Her2 IgGX-AF488 at 37° C. (or 4° C.) for 2 h (FIG. 4E, panels I, II, and IV). For FIG. 4E panel III, cells were further incubated with 1 μM (20-fold excess) of unlabeled anti-CD11a IgGX for 20 min followed by the 2 h incubation with 50 nM anti-CD11a IgGX-AF488 at 37° C. After treatment, all the non-internalized antibodies were removed by washing with acidic buffer (Tris-Glycine buffer, pH 2.5), and later neutralized with 1 M Tris-HCl (pH 7.5). After washing with PBS three times (with repeated centrifugation at 4° C.), the cells were fixed with 3.7% formaldehyde, followed by staining with Alexa Fluor 594-wheat germ agglutinin and Hoechst 33342 under manufacturer's instruction (Life Technologies). Visualization of samples was performed on a confocal laser-scanning microscope Zeiss 710 (Carl Zeiss).

### Example 12

### Mouse Cross-Reactive LXR-ADC for In Vivo Model

A mouse cross-reactive ADC based on a rat IgG2a anti-mouse CD11a monoclonal antibody is generated. In addition, an Fc-null anti-CD11a IgG with several mutations in the Fc region is constructed to prevent binding and internalization through Fcγ receptors and further improve selectivity and suitability for use in vivo.

What is claimed is:

1. An antibody drug conjugate comprising:
a. a liver X receptor agonist, and
b. an antibody that binds a cell surface molecule that is not expressed on a hepatocyte.

2. The antibody drug conjugate of claim 1, wherein the cell surface molecule is expressed on a macrophage.

3. The antibody drug conjugate of claim 1, wherein the cell surface molecule is CD11a.

4. The antibody drug conjugate of claim 3, wherein the antibody has a heavy chain variable domain having at least about 80% sequence identity to SEQ ID NO. 12.

5. The antibody drug conjugate of claim 1, wherein the antibody is chemically linked to (a) the liver X receptor agonist or (b) a moiety chemically attached to the liver X receptor agonist.

6. The antibody drug conjugate of claim 5, wherein the antibody comprises an unnatural amino acid chemically linked to (a) the liver X receptor agonist or (b) the moiety chemically attached to the liver X receptor agonist.

7. The antibody drug conjugate of claim 1, wherein the antibody drug conjugate has the structure I:

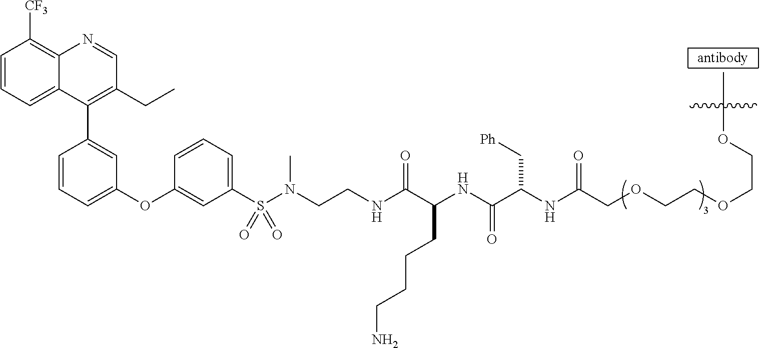

8. An antibody drug conjugate comprising:
a) a liver X receptor agonist, and
b) an antibody that binds to an integrin expressed on a surface of a leukocyte.

9. The antibody drug conjugate of claim 8, wherein the antibody has a heavy chain variable domain having at least about 80% sequence identity to SEQ ID NO. 12.

10. The antibody drug conjugate of claim 8, wherein the antibody is chemically linked to (a) the liver X receptor agonist or (b) a moiety chemically attached to the liver X receptor agonist.

11. The antibody drug conjugate of claim 10, wherein the antibody comprises an unnatural amino acid chemically linked to (a) the liver X receptor agonist or (b) the moiety chemically attached to the liver X receptor agonist.

12. A method for activating a liver X receptor in a leukocyte, the method comprising contacting the leukocyte with an antibody drug conjugate comprising:
a) a liver X receptor agonist, and
b) an antibody specific for a cell surface molecule of the leukocyte, wherein the cell surface molecule is not present on a hepatocyte.

13. The method of claim 12, wherein the cell surface molecule is an integrin.

14. The method of claim 12, wherein the antibody has a heavy chain variable domain having at least about 80% sequence identity to SEQ ID NO. 12.

15. The method of claim 12, wherein the liver X receptor comprises liver X receptor alpha, liver X receptor beta, or a combination thereof.

16. The method of claim 12, wherein the antibody is chemically linked to (a) the liver X receptor agonist or (b) a moiety chemically attached to the liver X receptor agonist.

17. The method of claim 16, wherein the antibody comprises an unnatural amino acid chemically linked to (a) the liver X receptor agonist or (b) the moiety chemically attached to the liver X receptor agonist.

18. The method of claim 17, wherein the antibody has a heavy chain sequence having at least about 90% sequence identity to SEQ ID NO. 10.

19. The method of claim 12, wherein the leukocyte is in a subject having a disease or condition selected from atherosclerosis, diabetes, inflammation, and Alzheimer's disease.

20. The method of claim 12, wherein the antibody drug conjugate has the structure I: [chemical structure]
